(19) United States
(12) Patent Application Publication
(10) Pub. No.: US 2011/0077215 A1
(43) Pub. Date: Mar. 31, 2011

(54) DNA Methylation As A Target For Diagnosis And Treatment Of Chronic Lymphocytic Leukema (CLL)

(75) Inventors: Margaret Yu, Salt Lake City, UT (US); John Phillips, Salt Lake City, UT (US)

(73) Assignee: THE UNIVERSITY OF UTAH RESEARCH FOUNDATION

(21) Appl. No.: 12/096,156

(22) PCT Filed: Dec. 7, 2006
(86) PCT No.: PCT/US06/46718

§ 371 Date: Nov. 12, 2008

Related U.S. Application Data
(60) Provisional application No. 60/749,323, filed on Dec. 7, 2005.

(51) Int. Cl. A61K 31/7076; A61K 31/7068; A61P 35/02

(52) U.S. Cl. 514/46; 514/49

(57) ABSTRACT

Global DNA methylation is a predictor of aggressive disease in patients with chronic lymphocytic leukemia. The higher the DNA methylation, the more likely a patient is going to require systemic therapy. Although there is a gradual decline in global DNA methylation with increasing age in normal individuals, the methylation index only decreases by approximately 0.03 per decade. A pilot study was performed in which patients with chronic lymphocytic leukemia were treated with low doses of DNA methylation inhibitors to evaluate if inhibition of DNA methylation can translate into a clinical benefit. Inhibition of DNA methylation was observed to lead to re-expression of tumor suppressors and normal cellular function. At low non-toxic doses of 0.05-0.09 mg per kilogram per day for three days every 28 days, some patients with chronic lymphocytic leukemia were observed to achieve a reduction in circulating leukemia cells. This was observed to correlate with a reduction in global DNA methylation and an alteration in methylation of core histones.

### CROSS-REFERENCE TO RELATED PATENT APPLICATIONS

This application claims the benefit of U.S. provisional application No. 60/749,323, filed on Dec. 7, 2005, which is incorporated herein by reference in its entirety.

### BACKGROUND

The present invention relates to the field of cancer. In particular, the invention relates to methods for treating, diagnosis, and/or obtaining a prognosis for patients with cancers such as leukemia.

Patients with leukemia such as chronic lymphocytic leukemia (CLL) are risk stratified based on various factors such as the mutational status of the immunoglobulin genes and karyotype abnormalities. For example, although CLL is an indolent disease, a subgroup of patients has aggressive disease. Patients found with the worst genomic aberration in CLL, a deletion in chromosome 17p, have a median survival of 32 months compared to a median survival of 133 months in patients with the best genomic aberration, a deletion in chromosome 13q. (See Dohner et al., N. ENGL. J. MED., (2000), 343:1910-16). Patients with a “normal” cytogenetic profile as defined by lack of deletions or duplications by fluorescence in situ hybridization, do not often have the best prognosis. The leukemic clones may not be truly normal, but continued growth could be driven by epigenetic modifications.

Like other solid organ malignancies, genomic DNA hypomethylation is observed in the peripheral blood mononuclear cells from patients with CLL. (See Wahlfors et al., BLOOD (1992) 80:2074-2080). Although DNA is globally hypomethylated, the promoters of selective tumor suppressor genes are often hypermethylated and silenced. For example, some patients with aggressive B-cell lymphomas or with low grade lymphomas that have transformed often contain tumor suppressors silenced by methylation. (See Fulop et al., LEUKEMIA (2003) 17:411-15; and Pinyol et al., BLOOD (1998) 91:2977-2984). Unlike mutations, silenced tumor suppressor genes could be reactivated by treatment with DNA methylation inhibitors. Therefore, DNA methylation levels may be correlated with aggressiveness of disease in patients with leukemia such as CLL, and if so, may be treated by administering methylation modulators.

### SUMMARY

Disclosed are methods for treating, diagnosing, and/or obtaining a prognosis for cancer in a patient. In particular, the methods relate to treating, diagnosing, and/or obtaining a prognosis for cancers in a patient such as leukemia, which may include leukemia of lymphoid origin (i.e., lymphocytic or lymphoblastic leukemia) or myeloid origin (i.e., myeloid or myelogenous leukemia). The leukemia may be chronic (e.g., chronic lymphocytic leukemia (CLL) or chronic myeloid leukemia (CML)) or acute (e.g., acute lymphocytic leukemia (ALL) or acute myeloid leukemia (AML)). Typically, the methods include determining whether leukemia cells of the patient exhibit DNA hypermethylation (e.g., global DNA hypermethylation). For example, the leukemia cells may exhibit an elevated percentage of methylated cytosine relative to total cytosine in genomic DNA when compared to normal cells.

In some embodiments, the determined percentage for a patient's leukemia cells may be compared to a percentage of methylated cytosine relative to total cytosine in genomic DNA of normal cells (e.g., normal cells of one or more individuals having similar demographic parameters such as age or sex). In some embodiments, the determined percentage for a patient's leukemia cells may be compared to a percentage of methylated cytosine relative to total cytosine in genomic DNA of normal cells in one or more individuals having an age that is relative close to the patient's age (e.g., within about 5 years of the patient's age). For example, the determined percentage may be compared to an expected mean for normal cells. In some embodiments, the determined percentage may be compared to an expected age-specific methylation index (“E”) calculated using the formula E=4.00−0.0034×A, where “A” is the age of the patient. In some embodiments, the determined percentage for a patient's leukemia cells may be compared to a percentage of methylated cytosine relative to total cytosine in genomic DNA of leukemia cells from one or more individuals having leukemia at the same stage or classification as the patient (e.g., based on the Rai, Modified-Rai, or Binet classification or staging systems).

The methods may be used to obtain a prognosis for a patient having leukemia. For example, the methods may be used to obtain a prognosis for a patient having an indolent, chronic leukemia (e.g., indolent CLL). In some embodiments, the methods may be used to predict whether a patient having indolent CLL is likely to progress to a more aggressive form of CLL. Based on the prediction, a treatment may be administered to prevent progression to the more aggressive form of CLL or treatment may be omitted.

In some embodiments, the methods include treating leukemia in a patient. For example, the methods may include methods of treating CLL in a patient (e.g., a patient having an indolent form of CLL). The methods may include: (a) determining a percentage of methylated cytosine relative to total cytosine in genomic DNA of leukemia cells of the patient; and (b) administering a treatment for leukemia (e.g., a treatment for CLL) to the patient based on the determination. Alternatively, the methods may include not administering a treatment for leukemia to the patient based on the determination. In some embodiments, the determined percentage may be compared to a percentage for normal cells from one or more individuals meeting a similar demographic parameter as the patient (e.g., an individual having an age within about 5 years of the patient or an individual having the same sex as the patient) or the determined percentage may be compared to an expected age-specific methylation index as described herein. The percentage may be determined and/or compared before, during, and/or after administering treatment. In some embodiments, treatment is administered where the patient's leukemia cells exhibit hypermethylation (i.e., a higher percentage of methylation than expected).

The methods may include performing additional determinations related to treatment, diagnosis, or prognosis for leukemia in a patient, before, during, or after administering a treatment to the patient as discussed below. For example, the methods may include determining a total white blood cell (WBC) count for the patient. In some embodiments, a treatment may be administered where the patient exhibits elevated global DNA methylation and elevated total white blood cell counts (e.g., at least about 5.0×10⁴ WBC/μl, or at least about 1.0×10⁵ WBC/μl, or at least about 1.5×10⁵ WBC/μl).

The methods may include determining whether the leukemia cells are positive for a marker such as Zap-70 or CD38 (e.g., by performing immunodetection or nucleic acid analysis) before, during, or after administering a treatment to the patient. The methods may include determining whether the leukemia cells have a chromosomal alteration, before, during, or after administering a treatment to the patient. For example, the methods may include detecting an alteration in chromosome 13q (e.g., a deletion) or an alteration in immunoglobulin heavy chain genes (e.g., a mutation).

The methods may include determining whether the histone proteins of the patient's leukemia cells are modified, before, during, or after administering a treatment to the patient. Modifications may include methylation (e.g., H3 lysine 9 methylation) and acetylation. For example, the methylation state of histone proteins (e.g., H3) may be assessed using immunodetection (e.g., using antibodies specific for methylated lysine residues). The methylation state of histones from leukemia cells may be compared to the methylation state of histones from normal cells.

The methods may include measuring expression or detecting expression of one or more micro RNAs (miRNAs) in leukemia cells of the patient, before, during, or after administering a treatment to the patient. For example, the methods may include performing a nucleic acid analysis to detect miRNAs (e.g., RT-PCR) and/or performing a microarray analysis. In some embodiments, the methods may include measuring expression or detecting expression of one or more miRNAs selected from the group consisting of mir-17-3p, mir-21, mir-29a, mir-29b, mir-29c, mir-30e, mir-104, mir-126, mir-128a, mir-130a, mir-141, mir-142-3p, mir-148a, mir-151, mir-199a, mir-199a*, and mir-301. The methods may include observing increased miRNA expression after treatment (e.g., with an inhibitor of DNA methylation) versus before treatment.

The methods may include determining whether the leukemia cells of the patient comprise one or more methylated tumor suppressor genes, before, during, or after administering a treatment to the patient. For example, the tumor suppressor gene may comprise a methylated promoter. Methylation of the tumor suppressor gene may be assessed by any appropriate assay (e.g., methylation specific PCR (MSP) analysis, and digestion analysis using methylation sensitive endonucleases). In some embodiments, the methods may include determining whether the leukemia cells comprise one or more methylated genes selected from the group consisting of p15, p16, p53, hMLH-1, MAGE-1, Twist2, Zap-70, CDH1, CDH13, DAPK, CRBP1, RARE, DLEU7, LEU1, LEU2, LEU5, KPNA3, CLLD6, CLLD7, CLLD8, ABL1, ATF2, BAGE, BRCA1, Calcitonin, CASP8, CASP9, CD14, CDC2, CDKN2A, CFTR, CIITA, COX2, Cyclin D2, DAPK, DAPK, DBCCR1, DBCCR1, E-CAD, E-CAD, ER, ER, FHIT, G6PD, G6PD, GAGE1, GAGE1, GATA-3, GATA-3, GLUT4, GLUT4, GPC3, GPC3, HIN-1, HIN-1, hMLH1, hMLH1, HOXA2, HOXA2, H-ras, hTERT, IFN, IGRP, IL-4, IRF7, JUNB, KIR2DL4, K-ras, LAGE-1, Maspin, MDR1, MGMT, MINT2, MINT31, MLC1, MT-X, MUC2, MYCL2, MyoD, NES-1, NF-1, NIS, NME2, NPAT, p21, p27KIP1, PAI-1, PAX6, PDGF-B, PgA, POMC, POU3F1, PR, Rb, RBL1, RIOK3, RPA2, SFN, SIM2, SRBC, STAT1, STATS, SYBL1, Tastin, TFF1, THBS1, THBS2, TIMP-3, TMS-1, TP73, and TSP-1. The methylated gene may comprise a methylated promoter.

The methods may include measuring beta-2-microglobulin or hemoglobin in the leukemia cells of the patient, before, during, or after administering a treatment to the patient.

Typically, the methods include administering a treatment (or alternatively, not administering a treatment), based on a global DNA methylation analysis of leukemia cells from a patient. A treatment may include administering at least one modulator of DNA methylation to the patient (e.g., an inhibitor of DNA methylation). For example, inhibitors of DNA methylation may include inhibitors of an S-adenosylhomocysteine hydrolase and inhibitors of an DNA methyltransferase (e.g., an inhibitor of at least one of DNA methyltransferase I, II, IIIa, IIIb, and IIIL). Inhibitors of DNA methylation may include nucleoside analogs or non-nucleoside analogs. In some embodiments of the methods, a patient is administered a nucleoside analogue having a modified cytosine ring that is attached to either a ribose or deoxyribose moiety. For example, nucleoside analogues may include cytidine analogues or deoxycytidine analogues that include a modification or substitution at the 5-carbon of the heterobase (e.g., 5′-azacytidine, 5′-aza-2-deoxycytidine(decitabine), 5′-fluoro-2-deoxycytidine, and 5′-chloro-2-deoxycytidine). Nucleoside analogues may include 5,6-dihydro-5-aza-cytidine and pyrimidin-2-one ribonucleoside(zebularine). In some embodiments, nucleoside analogues may include adenosine analogues or deoxyadenosine analogues. For example, nucleoside analogues may include adenosine analogues or deoxyadenosine analogues that include a modification or substitution at the 2-carbon of the heterobase (e.g., 2-carbon halo-substituted deoxyadenosine such as 2-chloro-deoxyadenosine(cladribine), 2-fluoro-deoxyadenosine(fludarabine), and cycloadenosine(aristeromycin)). The methods may include administering an inhibitor of S-adenosylhomocysteine hydrolase. For example, the methods may include administering a nucleoside analogue inhibitor of S-adenosylhomocysteine hydrolase (e.g., an adenosine analogue or deoxyadenosine analogue). The treatment may include administering at least one non-nucleoside analogue inhibitor of DNA methylation to the patient. Non-nucleoside analogues may include hydralazine, procainamide, EGCG, Psammaplin A, MG98 and RG108.

Typically, the methods include administering a treatment (or alternatively, not administering a treatment), based on a global DNA methylation analysis of leukemia cells from a patient. A treatment may include administering at least one modulator of histone methylation or histone deacetylation (e.g., an inhibitor of histone methylation or histone deacetylation). For example, the treatment may include administering short chain fatty acids (e.g., valproic acid and butyrate), hydroxamic acids (e.g., Trichostatin A (TSA) and suberoylanilide hydroxamic acid (SAHA), m-carboxycinnamic acid bishydroxamide, oxamflatin, scriptaid, pyroxamide, PDX-101, LBH589 and NVP-LAQ824), cyclic tetrapeptides (e.g., apicidin, depsipeptide, trapoxin, TPX-HA analogue (CHAP)), and benzamides (e.g., MS-275 and CI-994).

The methods may include administering cladribine. In some embodiments, the treatment comprises administering cladribine to the patient at a dose of about 0.05-0.1 mg/kg per day. In further embodiments, cladribine is administered to the patient for no more than about 5 consecutive days. The treatment may comprise a regimen whereby the patient is administered an initial dose (e.g., 0.05 mg/kg) and subsequently the dose is increased in increments of 0.01 mg/kg until the desired therapeutic effect is achieved. For example, a desired therapeutic effect may include a percentage reduction in total WBC count (e.g., at least about a 20% reduction, 30% reduction, 40% reduction, or 50% reduction). A desired therapeutic effect may include a percentage reduction in global DNA hypermethylation. A desired therapeutic effect may include detecting expression (or re-expression) of a miRNA (e.g., mi-34a or mi-195), after treatment (e.g., with a DNA methylation inhibitor).

Typically, the methods include administering a treatment to the patient (or alternatively, not administering a treatment patient) based on a determination related to global DNA methylation in leukemia cells of the patient. For example, the methods may include determining a percentage of methylated cytosine relative to total cytosine in genomic DNA of leukemia cells of the patient. In some embodiments, a treatment is administered if the determined percentage is at least about 3.7% (or 3.8%, 3.9%, 4.0%, or 4.1%). In further embodiments, the determined percentage is compared to an expected percentage for normal cells obtained from one or more individuals having an age within five (5) years of the patient, where treatment is administered if the determined percentage is higher than the expected percentage, otherwise treatment may not be administered. In even further embodiments, treatment may be administered if at least one of the following conditions is met: (a) the patient has an age of 0-9 and the determined percentage of methylation is at least 4.0%; (b) the patient has an age of 10-19 and the determined percentage methylation is at least 3.85%; (c) the patient has an age of 20-29 and the determined percentage of methylation is at least 3.94%; (d) the patient has an age of 30-39 and the determined percentage olmethylation is at least 3.90%; (e) the patient has an age of 40-49 and the determined percentage of methylation is at least 3.91%; (f) the patient has an age of 50-59 and the determined percentage of methylation is at least 3.82%; (g) the patient has an age of 60-69 and the determined percentage of methylation is at least 3.82%; (h) the patient has an age of70-79 and the determined percentage of methylation is at least 3.72%; (i) the patient has an age of 80-89 and the determined percentage of methylation is at least 3.71%; and (j) the patient has an age of 90-99 and the determined percentage of methylation is at least 3.63%. Where the methods include diagnosis, prognosis or treatment of CLL, typically the patient has an age that is at least about 40.

Typically, the methods achieve a desired therapeutic effect, including but not limited to a reduction in WBC count. For example, the methods may achieve at least about a 20% reduction in WBC count comparing level pre-treatment versus post-treatment (or at least about 30%, 40%, or 50%). The methods may achieve a desired therapeutic effect that include increase expression (or re-expression) of miRNA after treatment versus before treatment.

The disclosed methods also relate to methods for obtaining a prognosis for a patient having leukemia. For example, the disclosed methods may include methods for obtaining a prognosis for a patient having chronic lymphocytic leukemia (CLL). The methods typically include performing a global DNA methylation analysis, thereby obtaining a prognosis for the patient. In some embodiments, the method comprises determining a percentage of methylated cytosine relative to total cytosine in genomic DNA of leukemia cells of the patient. For example, where the leukemia cells of a leukemia patient are hypermethylated relative to normal cells (e.g., from one or more normal individuals), the prognosis may be for developing an aggressive form of leukemia. The disclosed methods also related to methods for diagnosing an aggressive form of leukemia (e.g., an aggressive form of CLL) in a patient, which may include determining a percentage of methylated cytosine relative to total cytosine in genomic DNA of leukemia cells of the patient.

### BRIEF DESCRIPTION OF THE DRAWINGS

FIG. 1 provides: A. DNA methylation levels in patients with CLL and B: proportion of patients not requiring treatment versus time (days).

FIG. 2 provides: A. patient characteristics at cladribine trial enrollment and B. Zap-70 expression in patients before treatment with cladribine.

FIG. 3 shows the analyses of five (5) patients under a Cladribine dose escalation regimen including global DNA methylation versus time (days) and total white blood cells (WBC) versus time (days). Patient #1 did not exhibit a decrease in hemoglobin (g/dL) over the course of the study.

FIG. 4 displays the results of a histone methylation analysis plotting fluorescence versus time. Fluorescently labeled anti-histone antibodies specific for methylated histone were used to detect methylated histone protein. A. Comparison of H3, H3 monomethyl K4, and H3 monomethyl K9. B. Comparison of H3 monomethyl K9, H3 dimethyl K9, and H3 trimethyl K9. C. Comparison of H3, H3 monomethyl K9, and H3 phosphoserine 10. D. Histone immunofluorescence of healthy volunteer sampled three times over 7 days.

FIG. 5 shows a scatterplot of measured methylation levels and white-blood counts.

FIG. 6 shows Kaplan-Meier curves for time until treatment by methylation index. Censoring times are jittered when several overlap. The p-value is for a log-rank test.

FIG. 7 shows three dose response curves: a linear, a concave, and a convex decreasing curve.

### DETAILED DESCRIPTION

The methods disclosed herein relate to the use of DNA methylation as a target for diagnosis, prognosis, or treatment of cancers such as leukemia. The methods may include performing a global DNA methylation analysis in order to assess whether to administer a systemic treatment to a leukemia patient.

Definitions

As used herein, the articles “a” and “an” refer to one or to more than one (i.e., to at least one) of the grammatical object of the article. By way of example, “an element” means one element or more than one element.

As used herein, the term “about” will be understood by persons of ordinary skill in the art and will vary to some extent on the context in which it is used.

As used herein, “leukemia” refers to a cancer of white blood cells, which may include leukemia of lymphoid origin (i.e., lymphocytic or lymphoblastic leukemia) or myeloid origin (i.e., myeloid or myelogenous leukemia). “CLL” refers to chronic lymphocytic leukemia involving any lymphocyte, including but not limited to various developmental stages of B cells and T cells, including but not limited to B cell CLL. As used herein the phrase “aggressive form of chronic lymphocytic leukemia” means that the subjects present, in addition to an abnormal hemogram, clinical symptoms such as an important tumor mass and/or cytopenia such as anemia or trombopenia. As used herein the phrase “indolent form of chronic lymphocytic leukaemia” means that subjects present an abnormal hemogram; however, do not present any clinical symptom normally associated with the disease. At the indolent stage, the disease is only detectable by labs means.

As used herein, “classification” and “staging” refers to well-known methods of leukemia classification or staging such as “Rai Classifications” and “Binet Staging.” Rai Classifications separates chronic lymphocytic leukemia into low-, intermediate-, and high-risk categories, which correspond with stages 0, I & II, and III & IV, respectively: Rai Stage 0 patients are low risk and have lymphocytosis, a high lymphocyte count defined as more than 15,000 lymphocytes per cubic millimeter (>15,000 /mm³). Rai Stage I patients are intermediate risk and have lymphocytosis plus enlarged lymph nodes (lymphadenopathy). Rai Stage II patients are also intermediate risk but have lymphocytosis plus an enlarged liver (hepatomegaly) or enlarged spleen (splenomegaly), with or without lymphadenopathy. Rai Stage III patients are high-risk and have lymphocytosis plus anemia, a low red blood cell count (hemoglobin <11 g/dL), with or without lymphadenopathy, hepatomegaly, or splenomegaly. Rai Stage IV patients are also high-risk but have lymphocytosis plus thrombocytopenia, a low number of blood platelets (<100−10³/μL). Binet Staging classifies CLL according to the number of lymphoid tissues that are involved (i.e., the spleen and the lymph nodes of the neck, groin, and underarms), as well as the presence of low red blood cell count (anemia) or low number of blood platelets (thrombocytopenia): Binet Stage A patients have fewer than three areas of enlarged lymphoid tissue. Enlarged lymph nodes of the neck, underarms, and groin, as well as the spleen, are each considered “one group,” whether unilateral (one-sided) or bilateral (on both sides). Binet Stage B patients have more than three areas of enlarged lymphoid tissue Binet Stage C patients have anemia plus thrombocytopenia (platelets <100−10³/μL).

As used herein, “DNA methylation” includes methylation of cytosine residues such as 5-methylcytosine. DNA methylation may include methylation of cytosine residues at CpG islands. The percentage of methylated DNA may be calculated by determining the amount of methylated cytosine in a sample (e.g., 5-methylcytosine) relative to total cytosine in a sample, where total cytosine is the sum of methylated cytosine and non-methylated cytosine. DNA methylation may include “global DNA methylation” which refers to DNA methylation throughout the cell genome. “DNA methylation” may refer to methylation of a specific target gene (e.g., at a CpG island within a target gene's promoter). .

As used herein, the term “gene” refers to a DNA sequence in a chromosome that codes for a product (either RNA or its translation product, a polypeptide). A gene contains a coding region and includes regions preceding and following the coding region (termed respectively “leader” and “trailer”). A gene includes a respective promoter sequence.

As used herein, the term “gene expression level” preferably means that the expression level has been quantitatively determined and is normalized.

As used herein, the phrase “patient” is defined as a subject that is suspected of having leukemia (e.g., CLL) or has been independently diagnosed as suffering from leukemia (e.g., CLL by conventional CLL diagnostic methods). Further, it should be noted that the term “patient” is used herein interchangeably with the term “subject.”

As used herein the term “sample” is defined as being any biological material naturally occurring or extracted in which cellular or genetic is contained. In an embodiment of the present invention, these terms refer to peripheral blood samples, tissue containing B cell, or extracted B cells. Within the context of the present invention, the specimen or sample may be used in a crude form, a preserved form (i.e., includes additional additives commonly added to preserve the integrity of the cellular material under environmental stress, such as freezing), a partially purified form, a purified form (e.g., isolated cellular material), or any other common preparatory form.

### Illustrative Embodiments

Embodiment 1. A method of predicting chronic lymphocytic leukemia (CLL) comprising examining the levels of global DNA methylation.

Embodiment 2. The method of embodiment 1, wherein increased global DNA methylation corresponds to an increased risk of the aggressiveness of CLL.

Embodiment 3. The method of embodiment 1, wherein global DNA methylation in excess of 3.77% indicates a greater need for treatment of CLL.

Embodiment 4. A method of limiting the progression of CLL in a patient comprising administering inhibitors of DNA methylation.

Embodiment 5. A method of preventing the development of aggressive CLL comprising administering an inhibitor of DNA methylation.

Embodiment 6. The method of embodiment 4 or 5, wherein the inhibitor is 2-chlorodeoxyadenosine(cladribine).

Embodiment 7. The method of embodiment 4 or 5, wherein the inhibitor is 5-aza-2-deoxycytidine(decitabine).

Embodiment 8. A composition that reduces global DNA methylation in order to limit the progression of CLL.

Embodiment 9. A composition that reduces global DNA methylation in order to prevent the development of CLL.

Embodiment 10. The composition of embodiment 8 or 9, wherein the inhibitor is 2-chlorodeoxyadenosine(cladribine).

Embodiment 11. The composition of embodiment 8 or 9, wherein the inhibitor is 5-aza-2-deoxycytidine(decitabine).

Embodiment 12. A method of screening for CLL comprising: (a) applying the composition of embodiment 10 or 11 to a biological system; and (b) assaying for a reduction in global DNA methylation.

Embodiment 13. A method for treating chronic lymphocytic leukemia (CLL) in a patient, comprising: (a) determining a percentage of methylated cytosine relative to total cytosine in genomic DNA of leukemia cells of the patient; (b) administering a treatment for CLL to the patient based on the determination.

Embodiment 14. The method of embodiment 13, further comprising determining a total white blood cell count for the patient.

Embodiment 15. The method of embodiment 13, further comprising determining whether the leukemia cells are positive for Zap-70.

Embodiment 16. The method of embodiment 13, further comprising determining whether the leukemia cells are positive for CD38.

Embodiment 17. The method of embodiment 13, further comprising determining whether the leukemia cells have a deletion in chromosome 13q.

Embodiment 18. The method of embodiment 13, further comprising determining whether the leukemia cells have a mutation in immunoglobulin heavy chain genes.

Embodiment 19. The method of embodiment 13, further comprising determining whether the leukemia cells comprise a higher proportion of methylated histone proteins relative to cells from a normal individual.

Embodiment 20. The method of embodiment 19, wherein the methylated histone proteins comprise methylated H3.

Embodiment 21. The method of embodiment 13, further comprising measuring expression of one or more micro RNAs (miRNAs) in the leukemia cells.

Embodiment 22. The method of embodiment 13, further comprising determining whether the leukemia cells comprise one or more methylated tumor suppressor genes.

Embodiment 23. The method of embodiment 13, further comprising determining whether the leukemia cells comprise a methylated gene selected from the group consisting of p15, p16, hMLH-1, MAGE-1, Twist2, Zap-70, CDH1, CDH13, DAPK, CRBP1, RAR□, DLEU7, LEU1, LEU2, LEU5, KPNA3, CLLD6, CLLD7, and CLLD8.

Embodiment 24. The method of embodiment 22 or 23, comprising determining whether the tumor suppressor gene has a methylated promoter.

Embodiment 25. The method of embodiment 13, further comprising measuring beta-2-microglobulin in the leukemia cells.

Embodiment 26. The method of embodiment 13, wherein the treatment comprises administering an inhibitor of DNA methylation.

Embodiment 27. The method of embodiment 26, wherein the inhibitor is an inhibitor of S-adenosylhomocysteine hydrolase.

Embodiment 28. The method of embodiment 27, wherein the inhibitor comprises cladribine.

Embodiment 29. The method of embodiment 13, wherein the treatment comprises administering cladribine at a dose of about 0.05-0.1 mg/kg per day for no more than about 5 consecutive days.

Embodiment 30. The method of embodiment 26, wherein the inhibitor comprises and inhibitor of a DNA methylatransferase.

Embodiment 31. The method of embodiment 30, wherein the DNA methyltransferase is DNA methyltransferase I or DNA methyltransferase Mb.

Embodiment 32. The method of embodiment 26, wherein the inhibitor is selected from the group consisting of 2-chlorodeoxyadenosine, 5′-azacytidine, 5′-aza-2′-deoxycytidine, and mixtures thereof.

Embodiment 33. The method of embodiment 13, wherein treatment is administered if the percentage is at least about 3.7%.

Embodiment 34. The method of embodiment 13, wherein treatment is administered if the percentage is at least about 3.8%.

Embodiment 35. The method of embodiment 13, wherein treatment is administered if the percentage is at least about 3.9%.

Embodiment 36. The method of embodiment 13, wherein treatment is administered if the percentage is at least about 4.0%.

Embodiment 37. The method of embodiment 13, wherein treatment is administered if the percentage is at least about 4.1%.

Embodiment 38. The method of embodiment 13, further comprising comparing the determined percentage to an expected percentage for cells obtained from one or more normal individuals having an age within five (5) years of the patient.

Embodiment 39. The method of embodiment 13, wherein treatment is administered if at least one of the following conditions is met: (a) the patient has an age of 0-9 and the determined percentage is at least 4.0%; (b) the patient has an age of 10-19 and the determined percentage is at least 3.85%; (c) the patient has an age of 20-29 and the determined percentage is at least 3.94%; (d) the patient has an age of 30-39 and the determined percentage is at least 3.90%; (e) the patient has an age of 40-49 and the determined percentage is at least 3.91%; (f) the patient has an age of 50-59 and the determined percentage is at least 3.82%; (g) the patient has an age of 60-69 and the determined percentage is at least 3.82% (h) the patient has an age of 70-79 and the determined percentage is at least 3.72%; (i) the patient has an age of 80-89 and the determined percentage is at least 3.71%; and (j) the patient has an age of 90-99 and the determined percentage is at least 3.63%.

Embodiment 40. The method of embodiment 13, wherein the method achieves at least about a 30% reduction in circulating leukemia cells.

Embodiment 41. The method of embodiment 13, wherein the method achieves at least about a 40% reduction in circulating leukemia cells.

Embodiment 42. The method of embodiment 13, wherein the method achieves at least about a 50% reduction in circulating leukemia cells.

Embodiment 43. A method for obtaining a prognosis for a patient having chronic lymphocytic leukemia (CLL), the method comprising determining a percentage of methylated cytosine relative to total cytosine in genomic DNA of leukemia cells of the patient.

Embodiment 44. The method of embodiment 43, wherein the prognosis is aggressive CLL.

Embodiment 45. A method for diagnosing an aggressive form of chronic lymphocytic leukemia (CLL) in a patient, comprising determining a percentage of methylated cytosine relative to total cytosine in genomic DNA of leukemia cells of the patient.

Embodiment 46. Use of an inhibitor of DNA methylation for treating chronic lymphocytic leukemia. (CLL) in a patient, wherein leukemia cells of the patient have an elevated percentage of methylated cytosine relative to total cytosine in genomic DNA when compared to normal cells from one or more individuals having an age within five (5) years of the patient.

Embodiment 47. Use of an inhibitor of DNA methylation for treating chronic lymphocytic leukemia (CLL) in a patient when at least one of the following conditions is met: (a) the patient has an age of 0-9 and leukemia cells of the patient have a percentage of methylated cytosine relative to total cytosine in genomic DNA of at least 4.0%; (b) the patient has an age of 10-19 and leukemia cells of the patient have a percentage of methylated cytosine relative to total cytosine in genomic DNA of at least 3.85%; (c) the patient has an age of 20-29 and leukemia cells of the patient have a percentage of methylated cytosine relative to total cytosine in genomic DNA of at least 3.94%; (d) the patient has an age of 30-39 and leukemia cells of the patient have a percentage of methylated cytosine relative to total cytosine in genomic DNA of at least 3.90%; (e) the patient has an age of 40-49 and leukemia cells of the patient have a percentage of methylated cytosine relative to total cytosine in genomic DNA of at least 3.91%; (f) the patient has an age of 50-59 and leukemia cells of the patient have a percentage of methylated cytosine relative to total cytosine in genomic DNA of at least 3.82%; (g) the patient has an age of 60-69 and leukemia cells of the patient have a percentage of methylated cytosine relative to total cytosine in genomic DNA of at least 3.82%; (h) the patient has an age of 70-79 and leukemia cells of the patient have a percentage of methylated cytosine relative to total cytosine in genomic DNA of at least 3.72%; (i) the patient has an age of 80-89 and leukemia cells of the patient have a percentage of methylated cytosine relative to total cytosine in genomic DNA of at least 3.71%; and (j) the patient has an age of 90-99 and leukemia cells of the patient have a percentage of methylated cytosine relative to total cytosine in genomic DNA of at least 3.63%.

Embodiment 48. Use of an inhibitor of DNA methylation for treating chronic lymphocytic leukemia (CLL) in a patient as recited in embodiment 46 or 47, wherein the inhibitor comprises an inhibitor of S-adenosylhomocysteine hydrolase.

Embodiment 49. Use of an inhibitor of DNA methylation for treating chronic lymphocytic leukemia (CLL) in a patient as recited in embodiment 46 or 47, wherein the inhibitor comprises 2-chlorodeoxyadenosine.

### EXAMPLES

### Example 1

### Global DNA Hypermethylation in Chronic Lymphocytic Leukemia Correlates with Progressive Disease

Global DNA methylation was assessed as a predictor of aggressive disease in patients with chronic lymphocytic leukemia (CLL). In addition, treatment with Cladribine was assessed as a potential DNA methylation inhibitor in CLL patients. Thirteen patients with chronic lymphocytic leukemia donated blood samples for DNA studies at the same time as blooddraws for their physician visits. Seventy-one percent (5/7) of patients with a global DNA methylation level above 3.8% required systemic chemotherapy within 8 months of assessment. One hundred percent (6/6) of patients below a DNA methylation level of 3.8% did not require systemic treatment for at least 2 years. Asymptomatic patients with chronic lymphocytic leukemia tend to have lower levels of global DNA methylation (median 3.5%) compared to symptomatic patients (median 4.5%). High levels of global DNA methylation (>3.8%) were associated with higher disease burden, corresponding with higher lymphocyte and white blood cell numbers. Five patients without immediate need for cytoreductive therapy were enrolled on a pilot treatment trial with low-dose cladribine, by subcutaneous injection. Three out of the five patients had a clinical response. Two of the patients achieved a partial response, as defined by the NCI-sponsored working group guidelines, with at least a three month follow-up after discontinuation of the drug. For two patients with stable or progressive disease, global DNA methylation levels of 3.7 and 3.8%, respectively, were observed, suggesting that higher DNA methylation levels correlate with more chemotherapy resistant disease.

### Example 2

### Global DNA Methylation Levels in Patients with Chronic Lymphocytic Leukemia and Modulation with Cladribine, a DNA Methylation Inhibitor

Materials

Gentra blood kits were used to extract DNA from peripheral blood mononuclear cells. S1 nuclease (Invitrogen, Calif.), phosphodiesterase I (Sigma, Mo.), alkaline phosphatase (Sigma, Mo.) were used in digesting genomic DNA extracted from peripheral blood mononuclear cells into nucleosides. 5-Methycytosine (Sigma, Mo.) and patient DNA were used as routine standards for each HPLC experiment. Antibodies used in immunochemical studies included anti-H3-mono-K9 (Novus Biologicals, Colo.), anti-H3-dimethyl-K9 (Novus Biologicals, Colo.), anti-H3-trimethyl-K9 (Novus Biologicals, Colo.), anti-acetylated-H3 (Novus Biologicals, Colo.), anti-H3 (Novus Biologicals, Colo.), fluorescein conjugated anti-sheep IgG (Rockland Immunochemicals, Pa.), fluorescein conjugated anti-rabbit IgG (Rockland Immunochemicals, Pa.), anti-Zap-70 (Upstate Cell Signaling Solutions, Va.), and goat anti-rabbit IgG-HRP (Santa Cruz Biotechnologies, Calif.).

Genomic DNA Digestion and HPLC Analysis

White blood cells were isolated from peripheral blood samples from patients with CLL using Ficoll-Hypaque. More than 95% of the white blood cells were CD5 and CD 19 positive by FACS analysis. DNA was extracted from the buffy using the Gentra blood kit. De-identified age-matched and gender-matched controls were obtained from Dr. Kang Zhang's database of healthy volunteers at the University of Utah. All DNA was adjusted to a final concentration of 200 μg/ml. Initially, 15 μg of DNA was fragmented into 200 bp-1000 by fragments for 60 seconds on ice. Samples were then denatured at 100° C. for 5 minutes and cooled on ice to prevent re-annealing. Sixty units of nuclease S1 and 112.5 mu of snake venom phosphodiesterase I in 12 μl of Si dilution buffer were added to the samples. They were then incubated at 37° C. for 18 hours. Samples were reheated to 100° C. for 5 minutes and cooled again on ice. Another sixty units of nuclease Si and 112.5 mu of snake venom phosphodiesterase I were added and incubated at 37° C. for another 4 to 6 hours. The pH of each sample was raised to 8.5 with 0.5 M Tris, pH 10. Two and a half units of alkaline phosphatase was added and incubated for 2 additional hours at 37° C. One hundred μl of 0.05M potassium phosphate, pH 7 was added to final samples before 50 μl of the clear supernatant was injected into the reverse-phase high performance liquid chromatography (HPLC).

A Phenomenex Gemini C18, 250×4.6 mm 5μ column on a reverse phase HPLC system consisting of an Alliance HT and a photodiode array detector was used to analyze individual nucleosides in the supernatant. Standards of 5-methyldeoxycytidine and deoxycytidine were used to quantitate the amount of each nucleoside in samples. The digested DNA was centrifuged at 13,000 rpm for 10 minutes. Each patient sample was digested in triplicate. Each digestion was injected twice. Error within digestion was less than 0.001% and the largest standard deviation between digestions was 0.2%. A linear gradient was made with 50 mM of potassium phosphate at pH 4.4 and HPLC-grade methanol. The sample eluted at 1 ml/min with deoxycytidine eluting at 10 minutes and 5-methyldeoxycytidine eluting at 18 minutes. The column was washed and recalibrated for 15 minutes following each sample. Absorbance of the eluent was monitored at 280 nm. Global DNA methylation was reported as a percentage of 5-methyldeoxycytosine relative to the sum of the 5-methyldeoxycytosine and deoxycytosine peaks.

Baseline Global DNA Methylation of Patients and Normal Controls

DNA from 23 patients with chronic lymphocytic leukemia has been collected from consenting patients from the outpatient hematology/oncology clinic at the University of Utah. One patient was excluded from the analysis secondary to inadequate followup. Nine were men. Many of these patients had been evaluated by the CLL FISH panel. Zap-70 expression was assessed by immunohistochemistry in some of the patients.

Phase I Dose-Escalation with Cladribine

Five patients with confirmed diagnosis of chronic lymphocytic leukemia by flow cytometry who did not require immediate cytoreductive therapy participated in this early phase clinical trial. Additional inclusion criteria included: KPS>70%, absolute neutrophil count >1500/μl, platelet count >100,000/μl, creatinine <2.0 mg/dl, normal liver function tests, no evidence of active infections on chest x-ray or urinalysis, and no concurrent chemotherapy or radiation therapy within 4 weeks of registration. Each patient signed an informed consent form approved by the University of Utah institutional review board, Clinical Cancer Investigation Committee, and Salt Lake City Veterans Administration Medical Center (SLC VAMC) Research and Development office.

The study accrued three patients from the VAMC and two patients from the Huntsman Cancer Institute at the University of Utah from January to May 2005. The patients were given three consecutive subcutaneous injections of cladribine (Leustatin) once every 28-32 days, starting at a dose of 0.05 mg per kg per day. The primary endpoint of the study was to find the dose of cladribine which caused a 20% decrease in global DNA methylation. Each patient served as his own control, and was dose-escalated every 28-32 days by 0.01 mg per kg per day if the primary endpoint was not met. Patients were given prophylactic doses of trimethoprim/sulfamethoxazole for the prevention of pneumocystis carinii. Patients allergic to trimethoprim/sulfamethoxazole were prescribed prophylactic doses of dapsone. No clinical endpoints were written into the study initially. The protocol was later modified to include a clinical stopping point after a clinical response was observed in three of the patients. A clinical response was defined by the NCI-sponsored working group for CLL. (See Cheson et al., BLOOD (1996) 87:4990-97). A patient was considered to have completed the study when the total white blood count was less than 10,000 cells/μl after two consecutive weekly blood counts. The entire study was conducted at the University of Utah General Clinical Research Center (GCRC). Leustatin was provided to the patients by Tibotec Therapeutics, a division of Johnson and Johnson.

The DNA and the mononuclear cells from the buffy coat were stored from the majority of patient samples. DNA was used for global DNA methylation studies and the mononuclear cells from the buffy coat were used to measure changes in H3 methylation by immunofluorescence before and after treatment.

Immunofluorescence

3×10⁴ CLL cells were mounted onto a slide by cytospin at 800 rpm for 3 minutes. They were then fixed in 95% ethanol/5% acetic acid for 10 minutes, followed by incubation in 0.25 M sodium butyrate for 24 hours at room temperature. The slides were stained by the Ventana automated immunostainer at 40° C. The slides were first stained with the primary antibody (1:50) for 32 minutes, followed by staining with the secondary antibody (sheep or rabbit IgG-FITC) (1:200) for 32 minutes. Slides were counterstained with DAPI (1:100) for 1 minute and hematoxylin (Ventana Medical Systems) for 4 minutes. Fluorescent detection was performed with the IView DAB detection kit (Ventana Medical Systems). Intensity of fluorescence was quantitated by a wide field fluorescent microscope and quantified by the Openlab modular imaging software. Signal to noise was optimized by the software.

Results

Baseline Global DNA Methylation of Patients and Normal Controls

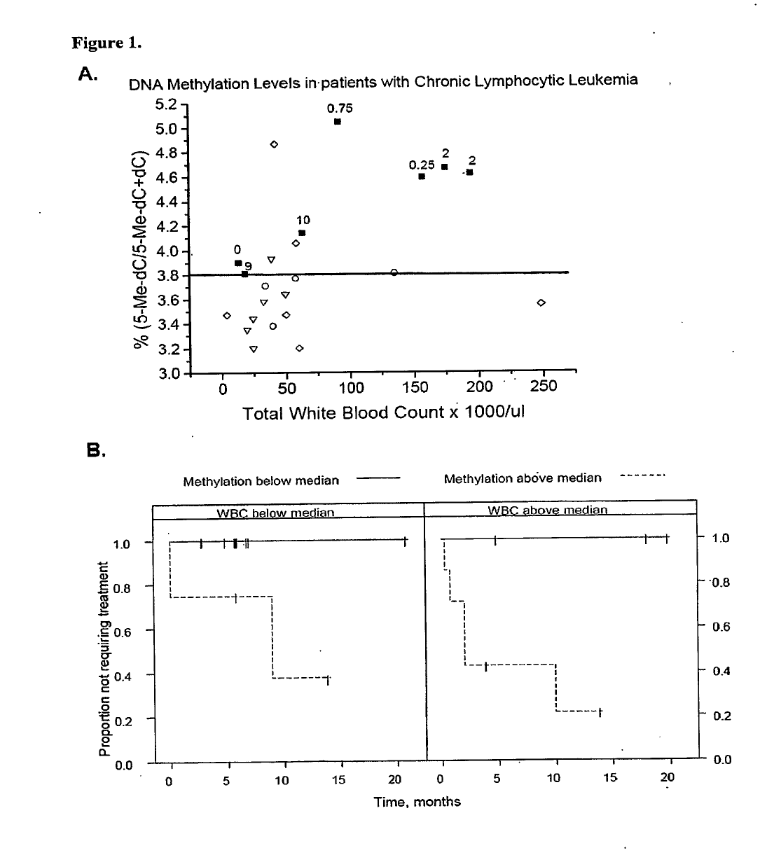

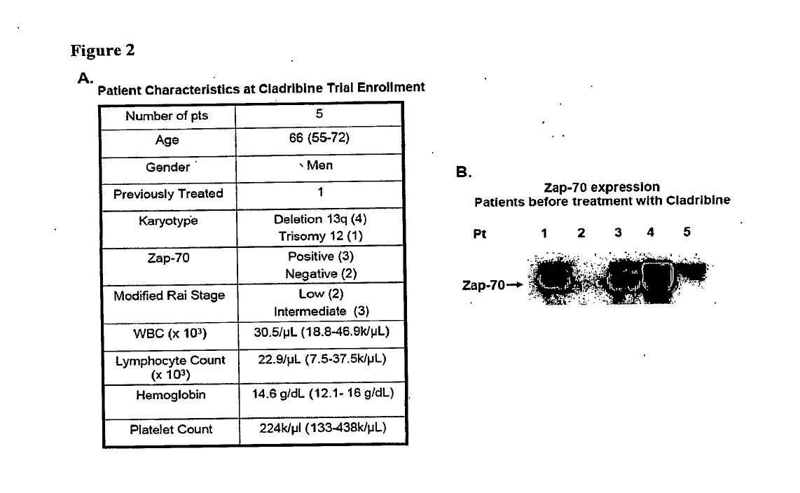

Global DNA methylation levels in asymptomatic patients with chronic lymphocytic leukemia were lower than normal, demographic and age-matched healthy controls (FIG. 1 and Table 1).

TABLE 1
Patient Characteristics

| | | | | | | | Follow- | | |
|---|---|---|---|---|---|---|---|---|---|
| | | %5-Me-dC/5- | | | Rai | | up | WBC | |
| Age | Sex | Me-dC+dC | Zap-70 | CD38 | Stage | FISH | (mos) | (10³/μL) | Tx |
| 51 | M | 5.045 | pos | pos | 4 | Del13q14 | .75 | 91.6 | Y |
| 73 | F | 4.865 | pos | n/a | 4 | n/a | 14 | 42.7 | N |
| 52 | F | 4.665 | pos | neg | 2 | Del13q14 | 2 | 174.8 | Y |
| 57 | M | 4.62 | neg | neg | 4 | Del13q14 | 2 | 194.0 | Y |
| 66 | M | 4.59 | pos | n/a | 4 | Del13q14 | .25 | 157.2 | Y |
| | | | | | | (5/05) | | | |
| | | | | | | Del17p | | | |
| | | | | | | (7/05) | | | |
| 67 | M | 4.14 | pos | pos | 4 | Trisomy12 | 10 | 62.9 | Y |
| 47 | M | 4.055 | neg | neg | 0 | n/a | 14 | 58.1 | N |
| 62 | M | 3.93 | n/a | n/a | 0 | n/a | 6 | 39.4 | N |
| 59 | M | 3.9 | neg | neg | 2 | 46XY | 0 | 13.2 | Y |
| 59 | M | 3.81 | n/a | n/a | 4 | n/a | 9 | 18.2 | Y |
| 80 | M | 3.79 | n/a | neg | 2 | Del13, | 4 | 134.8 | N |
| | | | | | | 14, 17 | | | |
| 71 | M | 3.77 | n/a | n/a | 2 | Trisomy12 | 5 | 57.4 | N |
| 57 | M | 3.71 | n/a | neg | 0 | Del13q14 | 3 | 34.2 | N |
| 78 | M | 3.64 | n/a | neg | 1 | Trisomy 12 | 7 | 49.7 | N |
| 86 | F | 3.58 | n/a | n/a | 0 | n/a | 7 | 32.8 | N |
| 64 | M | 3.55 | pos | n/a | 1 | Del13q14 | 18 | 248.8 | N |
| 70 | F | 3.47 | n/a | neg | 4 | Trisomy12 | 21 | 3.7 | N |
| 72 | F | 3.47 | pos | neg | 2 | n/a | 20 | 50.1 | N |
| 53 | M | 3.44 | n/a | n/a | 0 | n/a | 6 | 24.3 | N |
| 78 | F | 3.38 | n/a | neg | 2 | Normal | 5 | 40.0 | N |
| 90 | F | 3.35 | n/a | pos | 1 | Trisomy | 6 | 19.4 | N |
| | | | | | | 12/11q- | | | |
| 77 | M | 3.2 | n/a | n/a | 0 | n/a | 18 | 59.6 | N |
| 79 | M | 3.2 | pos | n/a | 0 | n/a | 6 | 24.2 | N |

However, DNA methylation was higher in patients who clinically required chemotherapy within a year of methylation measurement compared to healthy individuals (FIG. 1). Five out of six patients with global DNA methylation levels greater than 4.055 tested positive for Zap-70 (Table 1). Patients who required treatment presented with clinically worsening disease, evident either by worsening nightsweats, increasing circulating white blood cells, lymphadenopathy or organomegaly. Although there wasn't a target level which definitively correlated with more aggressive disease, higher levels within an individual did correlate with worsening disease. Patients were classified into two groups: those requiring treatment within 12 months and those not. Thirteen patients were used because at least 12 months of follow-up was available. The cutoff in methylation level providing the smallest observed prediction error is 3.795%; it correctly predicts 4/6 patients not needing treatment within a year and 7/7 patients needing treatment. These observed classification rates were adjusted for the bias resulting from the optimal selection of the cutoff using bootstrap. (See Efron and Robert J. Tibshirani. An introduction to the bootstrap, volume 57 of Monographs on Statistics and Applied Probability. Chapman and Hall, New York, 1993).

The adjusted sensitivity and specificity are 92% and 62%, respectively. The WBC cutoff providing the smallest observed prediction error is 62,150/μl; it correctly predicts 5/6 patients not needing treatment within a year and 5/7 patients needing treatment. The adjusted sensitivity and specificity are 63% and 80%, respectively. Thus both global DNA methylation and total white blood cell count appear to be valuable; perhaps a combination of high DNA methylation and high WBC would provide the best predictor.

Survival analysis was approached using the entire data set of 23 patients. Kaplan-Meier curves for above- and below-median methylation cutoff are shown. The accompanying p-value does not take into account the continuous nature of the methylation index, nor does it adjust for white blood count. Cox proportional hazard models were fitted to evaluate the effect of methylation as a continuous predictor, as well as to adjust for WBC levels. Based on the output shown in FIG. 1, the methylation level is a significant predictor of time to treatment, with an increase of one percentage point associated with a 6.1-fold increase in failure hazard (95% CI: 1.5-25). This effect is not affected by the inclusion of WBC in the model.

A gradual decline in global DNA methylation with increasing age is observed in normal individuals (Table 2).

TABLE 2
Global DNA Methylation Levels in Normal Individuals

| Decade | N | Mean * | Minimum | Maximum |
|---|---|---|---|---|
| 0 | 10 | 4.0 | 3.55 | 4.45 |
| 1 | 10 | 3.85 | 3.59 | 4.15 |
| 2 | 10 | 3.94 | 3.59 | 4.45 |
| 3 | 10 | 3.90 | 3.65 | 4.12 |
| 4 | 10 | 3.91 | 3.58 | 4.13 |
| 5 | 10 | 3.82 | 3.50 | 4.04 |
| 6 | 10 | 3.82 | 3.59 | 4.00 |
| 7 | 10 | 3.72 | 3.43 | 3.86 |
| 8 | 12 | 3.71 | 3.44 | 4.17 |
| 9 | 8 | 3.63 | 3.33 | 3.86 |
| Overall | 100 | 3.83 | 3.33 | 4.45 |

Global DNA methylation of approximately 10 healthy individuals per decade was measured. The methylation level of 100 healthy subjects of various ages (0-93.9) and of one control sample was assessed. The measurements were performed in 10 batches with the control sample present in each of them. The measurements on this control sample could be used to adjust for between-batch variability, however, since each batch contained a wide range of ages, the results of an analysis adjusting to the mean of each batch is very close to the one taking into account the control sample. Since the former is simpler to perform, the control sample was ignored in the following analysis. Each blood sample was digested twice and injected twice resulting in four measurements. Substantial variability was observed from subject-to-subject with smaller, but non-negligible variability between-digestion and between-injection. Mixed linear models were used to account for the (nested) random effects of batches, subjects, digestions, and injections. The computations were performed using the nlme library of R 2.1.0. (See Jose Pinheiro, Douglas Bates, Saikat DebRoy, and Deepayan Sarkar. nlme: Linear and nonlinear mixed effects models, 2005. R package version 3.1-57; and R Development Core Team. R: A language and environment for statistical computing. R Foundation for Statistical Computing, Vienna, Austria, 2005. ISBN 3-900051-07-0). Two models were used: one with a linear age effect and another with a flat age effect followed by a decrease. With the linear model, DNA methylation decreases by 0.03 per decade (p<0.00001). There was no evidence of non-linearity in the data set. Whether gender affects methylation levels was tested and no evidence of such an effect was found (p=0.68).

Phase I Cladribine Study

Five men with an average age of 66 were enrolled on the low dose cladribine study (FIG. 2A). None of these patients were included in the data set represented in FIG. 1. Patients 1, 2, 3, and 5 demonstrated a deletion in 13q14 by FISH. Patient 4 demonstrated duplication of 12. Patients 1, 3, and 5 also expressed Zap-70, assessed by western blot ((FIG. 2B). Most patients received at least three cycles of treatment. A total of 19 cycles were given. Patients 4 and 5 received all five dose escalations. Two grade II non-hematologic toxicities were observed (Table 3).

TABLE 3
Study Results

| | |
|---|---|
| Total number of cycles given | 19 |
| Number of cycles | 3-5 |
| CR | 0 |
| PR/Duration | 4/5 months |
| PD | 1 |
| Hematologic toxicities | Grade III/IV (0) |
| Non-hematologic toxicities | Grade I/II (2)* |
| | Grade III/IV (0) |
| Decrease in lymphocyte count | 48 days (8-67 days)** |
| by 50% | |
| Hemoglobin | 14.6 g/dL (12.1-16 g/dL)** |
| Platelet count | 224k/μL (133-438k/μL)** |

*Number of patients
**mean value (range of parameter of the 5 patients on trial)

Patients 1 and 2 had low risk disease and were asymptomatic. Patient 1 did not exhibit a decrease in hemoglobin (g/dL) over the course of the study. Patient 3 was previously treated, and had a resolving interstitial lung disease thought related to cyclophosphamide. He also had evidence of Sweet syndrome and a past history of fludarabine-induced hemolytic anemia. The fourth patient had never been treated, but had evidence of active disease with night sweats and mesenteric lymphadenopathy. Patient 5 had evidence of organomegaly, lymphadenopathy, and nonspecific symptoms of fatigue. He was otherwise asymptomatic. Patient 4 experienced grade II lymphadenitis, which resolved with oral antibiotics during the first cycle of cladribine. He subsequently experienced a second grade II infection, a community-acquired pneumonia 4 days following completion of cycle 5, which resolved after a course of oral antibiotics.

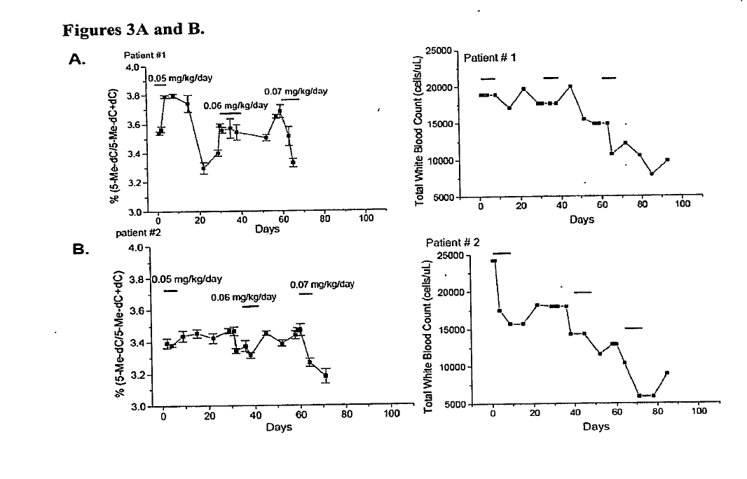

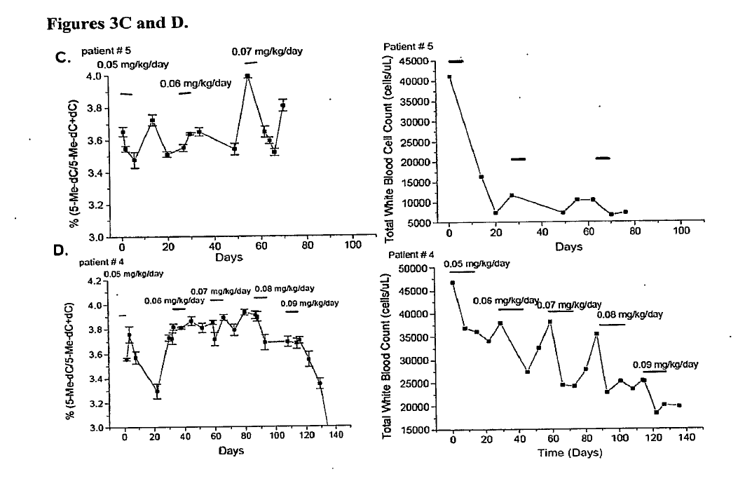

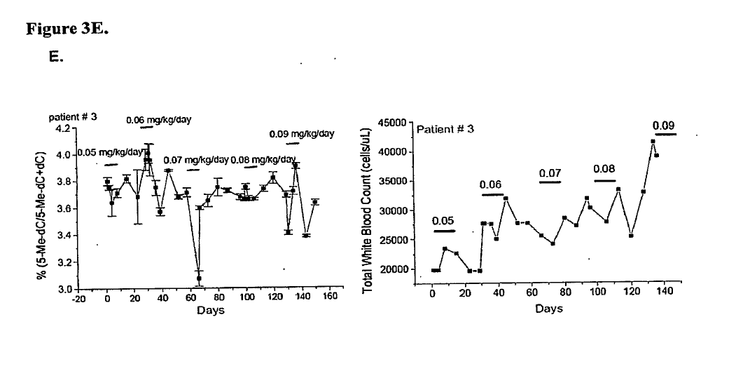

Four patients had partial responses and patient 3 demonstrated progressive disease (Table 3). Patient 3 had been previously treated with fludarabine, prednisone, and cyclophosphamide, whereas the rest of the patients had never received chemotherapy. Patients with low dose cladribine-sensitive disease had demonstrated a minimal (<10%) decrease in global DNA methylation, white blood count, without evidence of rebound, in contrast to minimally responding or progressing patients (FIGS. 3B and 3C vs. 3A, 3B, and 3E). Decreases in DNA methylation did not always correlate with a reduction in circulating white blood cell count (FIG. 3). In contrast, patient 3 demonstrated progression with a gradually increasing white blood count (FIG. 3E). He also demonstrated higher baseline levels of global DNA methylation compared to the others (FIG. 3E). His level of DNA methylation over time was consistent and minimally fluctuated throughout the study (FIG. 3E). Because there was a substantial variability between assays run on different days in terms of total DNA amount. Measurements with dC amount below 1000 or above 2500 were eliminated, as well as those done before

March 2005, or on Sep. 1, 2005. For examining pre-post treatment effects in each cycle, the day of measurement was selected as follows. The “pre” measurement was the one taken on the first day of the cycle (before the administration of Cladribine); if missing, it was substituted to the closest day with preference for an earlier date (in the previous cycle) in case of ties. The “post” measurement was the one taken 2 weeks after the first day of cycle; if missing, it was substituted to the closest day with preference for a later date.

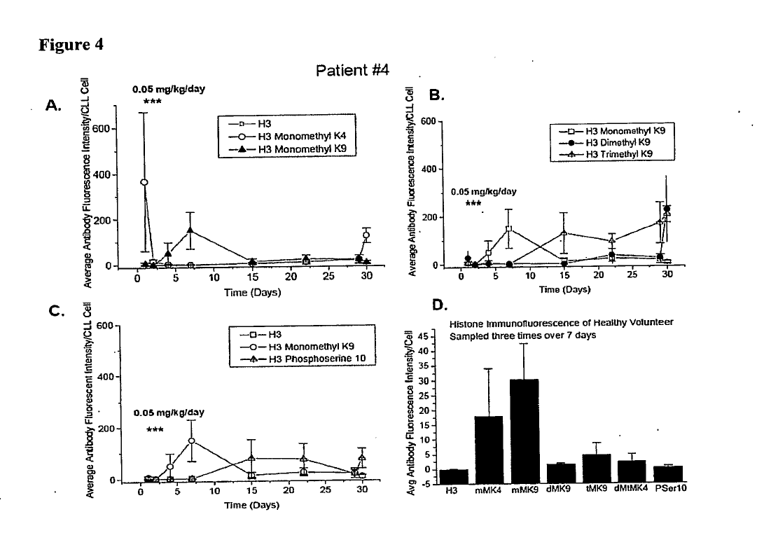

Patient 4 demonstrated a modest response in global DNA methylation, lymph node size, and white blood count after low dose cladribine. Peripheral blood mononuclear cells from patient 4 before and after treatment with 0.05 mg per kg per day of cladribine were assessed by immunofluorescence. Overall; minimal changes were seen in total histone 3. The average fluorescence intensity in monomethylated lysine 4 on the tail of histone 3 decreased to levels akin to signal intensity obtained from total histone 3, after 1 single low dose exposure of cladribine (FIG. 4A). There was an inverse relationship between monomethylated lysine 4 and monomethylated lysine 9 (FIG. 4A) as well as between monomethylated lysine 9 and phosphorylated serine 10 (FIG. 4C). An increase in monomethylated lysine 9 after low dose cladribine is followed by an increase in trimethylated lysine 9 approximately 1 week later (FIG. 4B). Little change was seen in dimethylated lysine 9. This is in contrast to the histone methylation pattern seen in healthy individuals, where levels of monomethylated lysine 4 and monomethylated lysine 9 are similar and stable over a period of a week (FIG. 4D). The error bars in FIG. 4 are not typical error bars as they correspond to the range of antibody immunofluorescence intensity in 100 cells.

Discussion

In this pilot study, low dose cladribine was demonstrated to induce a clinical response in some patients without toxicities. The dose response varied between patients and given the small sample size, it was not possible to predict the dose of drug that would work for everyone. For example, patient 5 had a dramatic reduction in white blood cell count following treatment with the first dose, at 0.05 mg per kg per day. Patient 1, on the other hand, did not demonstrate a reduction in circulating leukemia cells until the third dose escalation of 0.07 mg per kg per day. The decrease in circulating leukemia cells is very likely an effect from low dose cladribine, since a rebound in white blood cell count was generally observed prior to initiation of the next cycle of drug: One could argue that patients with sustainable decreases in circulating cells likely never required treatment anyway. On the other hand, if sustained normalization in the number of circulating CLL cells ultimately translates to prolonged disease free survival, low dose cladribine could be a non-toxic maintenance option. Long term follow up of treated patients will be required to definitively answer this question. Clinical response, defined by a greater than 50% reduction in circulating cells, seen in patients treated with cladribine was independent of a decrease in global DNA methylation level. This suggests that low dose cladribine (0.05-0.09 mg per kg per day given for 3 days every 28 days) may be clinically effective, but is not an effective DNA methylation inhibitor.

Reported is the first comprehensive global DNA methylation study of a range of patients with CLL compared to normal age and demographically matched controls. This is also the first analysis of global DNA methylation in patients with CLL correlated to time to treatment. Low dose cladribine (0.05 to 0.09 mg per kg per day for three days every 28 days) can induce a partial response without toxicities in patients not previously treated with Chemotherapy. This results in minimal inhibition in global DNA methylation, an increase in the monomethylated and trimethylated lysine 9 and a decrease in monomethylated lysine 4 and phospho-serine 10 on the tail of histone 3 in CLL cells. These chromatin changes correspond to a general state of transcription repression and gene silencing after low dose cladribine. (See Jaskelioff et al., NAT. CELL. BIOL. (2003) 5:395-399). Although the DNA methylation level is decreased in patients who normally do not require cytoreductive therapy, patients with more aggressive CLL have higher DNA methylation levels than normal age-matched controls. Confirmation of this observation will require methylation measurement of a larger population of patients with CLL.

### Example 3

### Histone Methylation Patterns After Treatment with Low-Doses of Cladribine

A histone methylation assay that uses immunofluorescence to assess for histone methylation patterns after treatment with low-doses of cladribine was developed. FIG. 4 shows the chromatin states over the course of a month, following treatment with cladribine. H3 monomethylated K4, a chromatin state normally associated with active transcription, was observed to decrease transiently after treatment with cladribine. This was accompanied by a rise in H3 monomethylated K9, H3 phospho S10, and H3 trimethylated K9. These states are associated with transcription silencing. (See Jaskelioff et al., Nat. Cell. Biol. (2003) 5:395-99). In FIG. 4, an average of 100 cells was quantitated in the fluorescence intensity at each time point. The asterisk corresponds to days of treatment with cladribine. The error bars at each time point refer to the range of antibody staining in all cells. Although the data shown in FIG. 2 represents an average fluorescence intensity of 100 cells, it was observed that all cells did not respond in a similar fashion. Multiparametric flow cytometry may be utilized to analyze further which subset of CLL cells respond to low doses of drugs.

### Example 4

### Baseline DNA Methylation in CLL Patients and Outcome

Classification Approach

Patients are classified into two groups: those requiring treatment within 12 months and those not. For 10 patients, this determination could not be made, so only 13 were used.

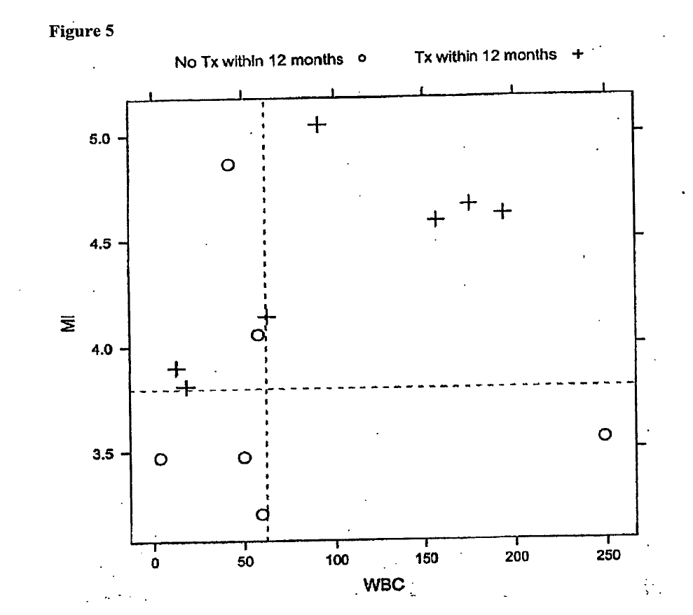

The cutoff in methylation level providing the smallest observed prediction error is 3,795%; it correctly predicts 4/6 patients not needing treatment within a year and 7/7 patients needing treatment. (See FIG. 5). These observed classification rates were adjusted for the bias resulting from the optimal selection of the cutoff using bootstrap. (See Efron and Robert J. Tibshirani. An introduction to the bootstrap, volume 57 of Monographs on Statistics and Applied Probability. Chapman and Hall, New York, 1993). The adjusted sensitivity and specificity are 92% and 62%, respectively.

For WBC, the cutoff providing the smallest observed prediction error is 62.15×10³ μl; it correctly predicts 5/6 patients not needing treatment within a year and 5/7 patients needing treatment. The adjusted sensitivity and specificity are 63% and 80%, respectively.

Thus, both predictors appear to be valuable; a combination of the two criteria: high methylation and high WBC may provide the best predictor.

Survival Analysis Approach

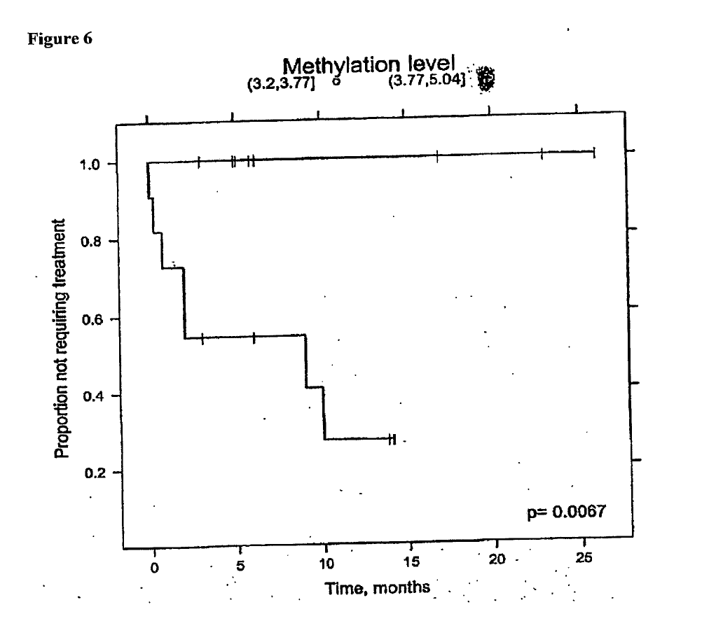

For the survival analysis approach, the entire data (23 patients) may be used. Kaplan-Meier curves for above- and below-median methylation cutoff are shown in FIG. 6. The accompanying p-value does not take into account the continuous of the methylation index, nor is it adjusted for white-blood count.

Cox proportional hazard models were fitted to evaluate the effect of methylation as a continuous predictor, as well as to adjust for WBC levels.

Based on the results in Table 4, the methylation level is a significant predictor of time to treatment, with an increase of one percentage point associated with a 6.1-fold increase in failure hazard (95% CI: 1.5-25).

TABLE 4
Results of Cox Regression Analysis

| | | | | |
|---|---|---|---|---|
| coef | exp(coef) | se(coef) | z | P |

Methylation only:

| | | | | | |
|---|---|---|---|---|---|
| MI | 1.85 | 6.34 | 0.694 | 2.66 | 0.0078 |

Likelihood ratio test = 7.62 on 1 df, p = 0.00578 n = 23

Methylation and WBC:

| | | | | | |
|---|---|---|---|---|---|
| MI | 1.81520 | 6.14 | 0.71704 | 2.532 | 0.011 |
| WBC | 0.00491 | 1.00 | 0.00555 | 0.885 | 0.380 |

Likelihood ratio test = 8.34 on 2 df, p = 0.0155 n = 23

This effect is not effected by the inclusion of WBC in the model.

### Example 5

### Global DNA Methylation, a Potential Predictor of Aggressive Chronic Lymphocytic Leukemia, and Cladribine as a Potential DNA Methylation Inhibitor

Global Methylation Analysis After Treatment with Decitabine

All patients were treated with continuous infusion Decitabine given intravenously in an inpatient setting. Comparison of patient DNA before and after treatment with Decitabine revealed a relative decrease in global DNA methylation. The method for assessing DNA methylation was consistent and reproducible (Samlowski et al. Manuscript submitted to JCO). By microarray analysis, decitabine was observed to upregulate many interferon inducible genes silenced by epigenetic alteration.

### Analysis of Candidate Tumor Suppressor Genes

Hypermethylation of candidate tumor suppressor genes may be assessed for use in diagnosing, providing a prognosis for, and/or treating CLL. For example, the methylation status of candidate tumor suppressor genes (e.g., p16, hMLH-1, and MAGE-1) may be assessed at the time of patient enrollment. Candidate tumor suppressor genes may be selected based on whether they have been found to be hypermethylated in aggressive B cell lymphoproliferative disorders. If a candidate tumor suppressor gene is found to be hypermethylated, then samples may be treated with an inhibitor of DNA methylation (e.g., Cladribine or Decitabine) and assessed for gene re-expression. Decitabine may be used as a positive control.

Evaluation of Global Methylation in CLL Patients Compared to Healthy Controls

Patient samples are stored at <−70° C. immediately after collection. Lymphocytes are collected from approximately fifty patients with CLL. Lymphocytes from participants are collected over a period of five-years from routine venopuncture. Leukocytes are separated from whole blood by ficoll-hypaque. As a primary endpoint, global DNA methylation status is correlated with the patient's stage of disease, total white blood cell count, LDH, and cytogenetics. Genomic DNA is isolated from blood samples and digested completely with nucleases and phosphatases. High performance liquid chromatography (HPLC) is used to fractionate nucleosides, and separate methyl cytosine from cytosine. The area under the trace may be used calculate the content of cytosine including methylated cytosine and non-methylated cytosine in the DNA.

Genomic DNA is treated with nuclease P1 (Sigma) and bacterial alkaline phosphatase (Sigma). Cell DNA (25 μg in 50 μL 10 mM Tris pH 7.2) is heated to 95 □C for 2 minutes, then immediately cooled on ice. Digestion with 2 units of nuclease P1 and 1.5 units bacterial alkaline phosphatase is performed at 37□C for 2 hours in 100 μL 30 mM sodium acetate buffer (pH 5.3) containing 5 μL 20 mM zinc sulfate. The mixture is heated and the pH adjusted by the addition of 20 μL 0.5 M Tris base for an additional 2-hour incubation at 37 □C. Samples are centrifuged at 20,000×g for 5 min and diluted in 50 mM potassium phosphate pH 7 containing 8-bromoguanosine (internal standard) to a final concentration of 9 μM. Samples (90 μL, derived from approximately 5 μg DNA) are fractionated with a Beckman System Gold HPLC and a Phenomenex Luna C18, 25×4.6 mm column, 126 NM solvent module, a 168 NM photodiode array detector and a 507e auto sampler using solvent A (2.5% MeOH/50 mM potassium phosphate pH 4.0) and solvent B (30% MeOH/50 mM potassium phosphate pH 4.0). The column is eluted at 1 ml/min beginning with 100% A for 10 minutes followed by a linear gradient over a period of 15 minutes to 100% B. After holding at 100% B for 10 minutes the column is recycled to 100% A with a linear gradient over a period of 10 minutes. Absorbance of the eluent at 200-300 nm is monitored, generating chromatograms that plotted absorbance at 284 nm (□max of 5-Me-dC at pH 4). The ratio of 5-Me-dC to dC is determined from a calibration curve resulting from the analysis of a serial dilution of nucleosides produced from digestion of normal human DNA. Samples are assayed in triplicate. Global DNA methylation is compared to DNA methylation levels expected from healthy controls. A decrease in global DNA methylation level of a least about 20% is sough, which is the level of decrease achievable in patients treated with Decitabine.

Global DNA-methylation will be analyzed on the logit-scale to stabilize its variance. DNA-methylation at enrollment will be connected to disease stage, white blood cell count and cytogenetics via regression to clarify the effect of these covariates. The within-patient longitudinal change in DNA-methylation will be modeled by a generalized linear model with auto-correlated errors. Sample size calculations for such methods are impractical, the calculation is based on a related, but simpler situation: the within-patient change in DNA-methylation level when the patient's disease progresses to a higher stage. A two-sided paired t-test at a 5% nominal significance level has an 80% power to detect a decrease in methylation from 5% to 4%, assuming a 1% patient-to-patient standard deviation with a sample size of 10. It may be expected that about 20% of the patients will experience disease progression during the study, thus approximately 50 patients may be monitored.

Asssessment of Candidate Tumor Suppressor Genes for Hypermethylation

Candidate tumor suppressor genes may be evaluated for hypermethylation. The basic methodology for assessing the MAGE-1 promoter is described below. Genomic DNA (4-10 μg) is isolated and digested with the methylation sensitive restriction enzyme, HpaII. Endonuclease-digested DNA will be amplified by polymerase chain reaction (PCR) using the CDS20, CDS21, and EPD4 primers for the MAGE promoter. The CDS20 primer lies 5□ to two Hpa II restriction sites within the MAGE-1 promoter CpG island, while the CDS21 primer lies 3□ to these two sites. EPD4 serves as the reverse primer for both CDS20 and CDS21 reactions. Therefore, the reaction containing the CDS20 primer interrogates the methylation level of these two sites, while the reaction containing the CDS21 primer serves as an internal control for DNA input. The DNA is amplified with PCR. The digestion is fractionated with electrophoresis on visualized by ethidium bromide staining. The same basic methodology may be used for other candidate tumor suppressor genes including p16 and hMLH-1, using appropriate primers for each gene.

Microarray Analysis of Gene Expression in Cells Treated with Cladribine

The effect of Cladribine on the re-expression of genes known to be silenced by methylation may be assessed, using Decitabine as an example. Both direct and indirect changes that result from reversal of the epigenetic silencing of gene expression may be assessed.

Lymphocytes are inclubated with 0-3 μM Cladribine. Total RNA is isolated and the effect of Cladribine on gene expression is assessed at intervals of 0, 24, 48, 96 and 120 hrs.

Total RNA is isolated with RNeasy mini kits (Qiagen) from samples treated with Cladribine or vehicle. Approximately 1 μg may be amplified using Message Amp kits (Ambion). In replicate experiments, DNA from the vehicle or treated samples may be variably labeled with Cy-3 and Cy-5 dye and two microarray experiments may be performed for each dye-orientation. The utilized microarray slides consist of 9600 genes from the Research Genetics 40k sequence-verified human clone set. Each sample is deposited in duplicate, with a Lucidea (Amersham) robotic spotter. After hybridization, the microarray slides are scanned with an Axon scanner, and data quantified using Imagene and GeneSight software. The local background is subtracted. Data points with no signal, high background, or spot asymmetry are eliminated. Genes with low expression and low signal intensity are adjusted to a minimal raw value of 5 to avoid unwarranted mathematical distortions due division by decimals <<1. After calculating the ratio of the Cy5/Cy3 fluorescence signal intensity for each gene, data is normalized relative to the mean intensity from all genes. Values from replicate clones are averaged, and the data transformed to log₂ each independent clone. Data is analyzed with Spotfire software for statistical and hierarchical cluster analysis using an unweighted pair-group method with arithmetic mean with a Euclidean distance metric.

The microarray experiment comparing Untreated, Decitabine- and Cladribine-treated samples will be set up in a loop design: Untreated (Cy3) vs Decitabine (Cy5), Decitabine (Cy3) vs Cladribine (Cy5) and Cladribine (Cy3) vs Untreated (Cy5). The gene expression data will be analyzed within the Significance Analysis of Microarray (SAM) framework [Tusher, 2001]. The data will be adjusted according to the loess-smoothing method of [Yang, 2000] and adjusted log-expression (not ratios) will be analyzed. Gene-specific ANOVA models will be used to obtain the appropriate F-statistic for the effect of interest (adjusted for the experimental design), followed by stabilization with an additive constant in the denominator. The main emphasis in the significance testing will be the estimation of the false discovery rate (FDR), that is the expected proportion of genes in a given list that are not differentially expressed. Using a DNA microarray analysis, re-expression of silenced genes in HT-29 cells (a colon cancer cell line) was observed after treatment with Cladribine.

Dose-Escalation Trial of Cladribine as a DNA Methylation Inhibitor

Dose-escalation trials may be performed to determine the minimal dose of Cladribine required to inhibit DNA methylation without causing significant cytotoxicity in CLL patients. For example, an initial dose of 0.05 mg/kg may be escalated by 0.01 mg/kg increments.

Cladribine is delivered subcutaneously at 0.05 mg/kg. (50% of current treatment dose) per day for three days at a time. Each patient will serve as his own control as weekly blood samples are compared to the initial blood sample prior to treatment with Cladribine. If no change in methylation is seen at this dose, dose is escalated by 0.01 mg/kg until arriving at a dose sufficient to cause inhibition of methylation compared to pre-treatment blood samples. Patients will not be dose escalated beyond 0.1 mg/kg, as cytotoxicity may be seen at this dose. Treatments cannot be given more frequently than allowed by hematologic parameters. Blood counts are monitored weekly. Adequate hematologic parameters and patient performance status have to be met before initiation of subsequent infusions of Cladribine. Patient blood samples will be drawn before the drug is given as well as after each 3 days of treatment. Enrolled patients and their blood samples will be evaluated every 7 days for re-methylation following the infusion to help identify an effective schedule of drug administration and to monitor for adverse events. Patients will be monitored using the NCI Common Toxicity Criteria.

Lymphocytes are separated from whole blood by the procedure described above. Genomic DNA methylation level and candidate tumor suppressor genes are assessed as described above, both before and after treatment with Cladribine. Statistical Analysis is described below.

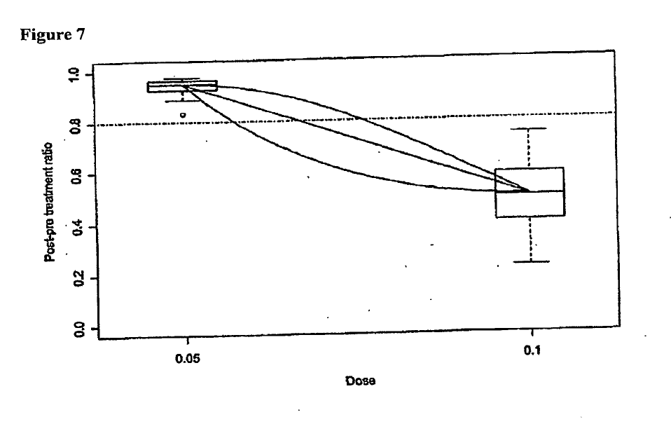

The objective is to estimate the dose of Cladribine that provides a20% decrease in global DNA methylation. A mixed linear model is fitted with patient-specific random effect to the observed post-pre treatment log-ratios of the DNA methylation level. The fitted model will be used to estimate the dose required for a 20% decrease (that is a ratio of 0.8), denoted by ED₈₀. Since the dose escalation within each patient will be stopped after observing an at least 20% decrease, the properties of the estimator of the ED₈₀ are intractable theoretically. Simulation are conducted to investigate the bias and precision of the estimator. It is assumed that each patient has an individual dose-response curve dependent on his/her true response (post-pre-treatment log-ratio of DNA methylation) at the minimum dose 0.05 mg/kg/d and maximum dose 0.1 mg/kg/d. In the population, the logit-transformed response is set at the minimum dose to be have a normal distribution with mean logit(0.95) (5% decrease in methylation) and standard deviation of 0.5, and at the maximum dose to have a mean of logit(0.5) and same standard deviation. Three dose response curves are analyzed: a linear, a concave and a convex decreasing curve. (See FIG. 7).

Based on previous experience with Decitabine, it may be estimated that the measurement of global DNA-methylation log-ratio has a standard deviation of 0.05. Based on these parameters, 10,000 sets of 5 patients were simulated following the protocol. The estimate of ED₈₀ has a bias not exceeding 0.0015 mg/kg/d and standard deviation of about 0.0035 mg/kg/d. These values are lower than those of two other methods of estimation considered: taking the mean of the largest dose archived by each patient and fitting an individual linear model for each patient and averaging the resulting personal estimates of ED₈₀. Use of 5 patients may be sufficient to estimate the dose of Cladribine resulting in 20% reduction of DNA methylation with precision of two digits after the decimal point.

### Example 6

### DNA Methylation Inhibitors Upregulate miRNAs in Patients with CLL

Genomic DNA Digestion and HPLC Analysis

White blood cells were isolated from peripheral blood samples from patients with CLL and healthy volunteers using Ficoll-Hypaque. {Wahlfors, 1992 #2} Most of the patients had more than 90% lymphocytosis. All DNA was adjusted to a final concentration of 200 μg/mL. Initially, 15 μg of DNA was fragmented into 200 bp-1000 by fragments by sonication for 60 seconds on ice. Samples were then denatured at 100° C. for 5 min and cooled on ice to prevent re-annealing. Sixty units of nuclease S1 and 112.5 mu of snake venom phosphodiesterase I in 12 μL of S1 dilution buffer were added to the samples. Samples were then incubated at 37° C. for 18 h and then reheated to 100° C. for 5 min and cooled again on ice. Another sixty units of nuclease S1 and 112.5 mu of snake venom phosphodiesterase I were added and incubated at 37° C. for another 4 h. The pH of each sample was raised to 8.5 with 0.5 MTris, pH 10. Two and a half units of alkaline phosphatase was added and incubated for 2 additional h at 37° C. One hundred μL of 0.05 M potassium phosphate, pH 7 was added to final samples. The digested DNA was centrifuged at 13,000 rpm for 10 min. Error within digestion was less than 0.001% and the largest standard deviation between digestions was 0.2%.

Fifty μL of the clear supernate was injected into an Alliance HT HPLC system equipped with a photodiode array detector. A Phenomenex Gemini C18, 250×4.6 mm 5μ column was employed and developed with a linear gradient of 50 mM of potassium phosphate at pH 4.4 and HPLC-grade methanol. Nucleosides were eluted at 1 mL/min with deoxycytidine eluting at 10 min and 5-methyldeoxycytidine eluting at 18 min. The column was washed and reequilibrated for 15 min following each sample. Absorbance of the eluent was monitored at 280 nm. Global DNA methylation was reported as a percentage of 5-methyldeoxycytosine relative to the sum of the 5-methyldeoxycytosine and deoxycytosine peaks. Standards of 5-methyldeoxycytidine and deoxycytidine were used to quantify the concentration of each nucleoside in the samples.

Baseline Global DNA Methylation of Patients

Peripheral blood mononuclear cells from men and women with CLL were collected from consenting patients from the Hematologic Malignancy clinic at the Huntsman Cancer Institute at the University of Utah. Many of these patients were also evaluated by the CLL FISH panel, which consisted of probes made for ATM, D12Z3, RB-1, D13S25, and p53. (ATM probes were used for deletion in 11q-; D12Z3 probes for trisomy 12; RB-1 and D13S25 probes for deletion in 13q-; p53 probes for deletion in 17p.) These probes were developed and validated by the University of Utah Cytogenetics laboratory. {Goorha, 2004 #47} Genomic DNA extraction was performed by the University of Utah General Clinical Research Center DNA core laboratory.

Effect of Low-Dose Cladribine In Vivo

Five patients with CLL were given subcutaneous injections of cladribine (Leustatin, provided by Tibotec Therapeutics) on three consecutive days at 28-32 day intervals, starting at a dose of 0.05 mg/kg/day, approximately ⅓ of the recommended therapeutic dose. {Juliusson, 1993 #10; Juliusson, 1996 #11} Five patients with a confirmed diagnosis of CLL (Table 3) participated in an Institutional Review Board-approved protocol between January and May 2005 after informed consent was obtained. All were clinically stable and not in need of treatment. Inclusion criteria included: KPS>70%, absolute neutrophil count >1500/μL, platelet count >100,000/μL, creatinine <2.0 mg/dL, normal liver function tests, no evidence of active infections, and no concurrent chemotherapy or radiation therapy within 4 weeks of registration.

The DNA MI was measured in each patient before and after treatment with low dose cladribine. In two of the patients, sufficient cells (>5×10⁶) remained to extract RNA for miRNA expression.

Effect of Low-Dose 5-Azacytidine In Vivo

Patients with CLL not in immediate need for cytoreductive therapy are enrolled on a dose-escalation study of low-dose 5-azacytidine (Vidaza, provided by the Pharmion Corporation.) Patients must have a KPS greater than 70%, white blood cell count greater than 20,000/μL hemoglobin >8 g/dL, platelet count >50,000/μL, normal liver, and renal functions. The study was carried out in 2 stages using a modification of the design proposed by Storer. {Storer, 1989 #16} In the first stage, patients were enrolled as single patient cohorts until either at least a 20% DNA MI inhibition or a greater than grade II toxicity. This allowed us to get closer to the target biologically effective dose before enrolling three patient cohorts. The global DNA MI and the miRNA expression profile of 6 patients before and after 5-azacytidine in the patients in the first stage of the study protocol was determined. Patients received 10-70 mg/m²/day on days 1-3 and days 8-10.

MiRNA Expression Profile of Patients with CLL

Total RNA is extracted with Trizol, as previously described. {O'Donnell, 2005 #182} MiRNA expression was determined using the Applied Biosystems Taqman MicroRNA Assays Human Panel Early Access Kit. 10 ηg of RNA is used for each sample. cDNA is made using a looped RT primer provided by the kit. A 1 to 5 dilution of the cDNA is used for each sample and used for quantitative PCR on the ABI 7900HT Fast Real-Time PCR System. MiRNA-specific forward and reverse primers were provided in plates A and B. MiR-16 and let-7a served as positive controls while let-2 served as a negative control for human samples. Each miRNA were performed in quadriplicates. The miRNA expression was determined for CD5⁺B-lymphocytes obtained from pediatric tonsils and from B-lymphocytes of 5 healthy controls.

Bisulfite Sequencing of Two miRNA Promoters

Putative promoters were identified using the UCSC genome browser. Two CpG islands to miR-34a and miR-195 were identified, both upregulated after cladribine and 5-azacytidine in patients with decreased lymphocytosis following drug treatment. Bisulfite conversion of genomic DNA was performed using Zymo Research EZ DNA Methylation-gold kit. Primers used to amplify bisulfite converted DNA were designed using Methyl Primer Express, version 1, software from Applied Biosystems. Primers were synthesized by the University of Utah DNA/peptide synthesis core laboratory.

PCR Conditions

PCR amplification was performed under the following cycling conditions: initial denaturation at 95° C. for 5 min, denaturation at 95° C. for 30 sec, annealing at 55° C. for 30 sec and extension at 72° C. for 30 sec, followed by a final extension step at 72° C. for 4 min using a MJ Research PTC-200 Peltier Thermal Cycler (Maryland, USA). Reactions were carried out in a 50 μL final volume, containing 2 Units of Platinum Taq DNA polymerase (Invitrogen, Carlsbad, Calif., USA), 1× of PCR reaction buffer, 1.5 mM MgCl₂, 10 mM dNTPs and 10 mM forward and reverse primers.

TABLE 5
Primer Sets for miR-195

| MiR- | | Forward Primers | Reverse Primers |
|---|---|---|---|
| 195 | Size | (5′ to 3′) | (5′ to 3′) |
| AB | 281 bp | GGAGGAGGTTAAAGGTTTTAGT | AATCCAAAAAAATAAACTCCC |
| CD | 258 bp | AGGATAATGGAAGGAAATTAATTAGAA | AACCAACCTTTACTACAAAACAAAAT |
| EF | 435 bp | TTTGTTTTGTAGTAAAGGTTGGTTT | TCTAACTCCCTCAATCTCTTATTCT T |
| GH | 261 bp | GGAGGAAGGAAGGATATAGATTTAG | AATCCCTAATTTAACCCAATTTCTA |
| IJ | 302 bp | TAGGAGTTTGTGTGGAGTTTGT | AACCAATTAAAAATTCCACACC |
| KL | 269 bp | GAGGTAGGGTTGGATATTTGAG | CAACTAAAACCAAAACTTCCAAC |
| MN | 391 bp | GGAAAGGGGTGGGATTATA | AACCACATACTCCATCTACAAA |
| QR | 355 bp | GGTTGGAAGTTTTGGTTTTAGTTGTA | ACCCTATAATCCCACCCCTTT |

For miR-34a, a different set of conditions were used. PCR amplification of the GC-rich region of miR-34a was performed under the following cycling conditions: 1 min initial denaturation step at 95° C. followed by 35 cycles at 94° C. for 30 sec, 50° C. for 30 sec and 72° C. for 60 sec, followed by a final extension step at 72° C. for 5 min using a MJ Research PTC-200 Peltier Thermal Cycler (Maryland, USA). The PCR reaction contained approximately 500 ng template DNA, 1×NH₄ of reaction buffer, 1.5 mM MgCl₂, 200 μM of each dNTP, 2 Units of Taq DNA polymerase (Biolase, Bioline Inc., Reno, Nev., USA) and 10 mM of each primer in a 50 μL final volume. The enhancing agent Betaine was added to PCR reactions to a final concentration of 1.3 M to improve amplification and stabilization of the DNA secondary structure.

TABLE 6
Primer Sets for miR-34a

| MiR- | | Forward Primers | Reverse Primers |
|---|---|---|---|
| 34a | Size | (5′ to 3′) | (5′ to 3′) |
| MN | 286 bp | GAGTAGGTAGTGTAGGTTTTTAGGT | TAATCCTCTTTCCTTTTCAAAT |
| OP | 425 bp | GATTAGGTGGGGGTTAGGTA | CTATTTACTTTTATACAATCCCCC |
| QR | 450 bp | GGGTTAGGATTTGTTGTTTTTG | CCATACTCCCCCCTATAACTC |
| ST | 304 bp | AGTTATAGGGGGGAGTATGGG | AAAAACCCTTACAAAAAAAACCAC |
| UV | 316 bp | GTGGTTTTTTTTGTAAGGGTTTTT | ACCCCTAACCTCCTCTCAAA |

PCR products were then cloned using the Invitrogen zero blunt end TA cloning kit.

Results: Baseline Global DNA Methylation of Patients with CLL

Effect of Low-Dose Cladribine In Vivo

Five men, with an average age of 66, were given low-dose cladribine to study the effect of cladribine on DNA methylation. Three of the five patients expressed Zap-70 by immunochemistry (FIG. 2). Three patients received three cycles of treatment with cladribine. Patients 4 and 5 received all five dose escalations. Patients 1 and 2 were asymptomatic, and had received no prior therapy. Patient 3, the only patient who was previously treated, had a resolving interstitial lung disease thought related to cyclophosphamide. He also had evidence of Sweet syndrome and a past history of fludarabine-induced hemolytic anemia. The fourth patient had no prior treatment, but did have night sweats and lymphadenopathy. Patient 5 had no prior treatment, but had organomegaly, lymphadenopathy, and the nonspecific symptom of fatigue. Patient 4 experienced grade II lymphadenitis, which resolved with oral antibiotics during the first cycle of cladribine. He subsequently experienced a second grade II infection, a community-acquired pneumonia 4 days following completion of cycle 5, which resolved after a course of oral antibiotics. Patients 1, 2, 4, and 5 had a DNA MI of 3.54, 3.39, 3.56, and 3.65, respectively, prior to initiating treatment with low dose cladribine. All of them demonstrated at least a 50% reduction in white blood cell count that lasted for 5 to 13 months after treatment with low dose cladribine. Patient 4 had a trisomy 12 while the rest of the patients had a deletion in chromosome 13q14 as the only karyotype abnormality. Less than 10% transient decrease in DNA MI was noted in these patients (FIG. 2). In contrast, patient 3, had a DNA MI higher than expected for his age before treatment and demonstrated progression with a gradually increasing white blood count. He developed a deletion in chromosome 17p while on therapy.

Effect of Low-Dose 5-Azacytidine In Vivo

Five men and one woman with an average age of 79 were given low-dose 5-azacytidine to study the effect 5-azacytidine on DNA methylation in patients with CLL. The first patient had normal cytogenetics by FISH and by routine karyotyping. The second, fourth, and fifth patients had a deletion in 13q14 as the sole genetic abnormality. The third patient had a deletion in chromosome 17p. The sixth patient had a trisomy 12. Four out of the 6 patients demonstrated approximately a 10% decrease in global DNA MI. No change in global DNA MI was observed in patients 2 and 4 after low-dose 5-azacytidine. Unlike patients treated with cladribine, trends in global DNA MI did not correlate with total white blood cell count. The 5^{th} patient, on a dose of 60 mg/m²/day, experienced grade II thrombocytopenia and anemia. No other toxicities were observed in the other patients.

### Example 7

### DNA Methylation Inhibitors Upregulate miRNA Expression in Patients with CLL

Re-expression of miRNA by 5-azacytidine and 2-chloro-2-deoxyadenosine

Two different DNA methylation inhibitors were studied with respect to re-expression of microRNA: a DNA methyltransferase enzyme inhibitor, 5-azacytidine, and a methyl substrate inhibitor, 2-chloro-2-deoxyadenosine. Increased microRNA expression was observed in six patients treated with 5-azacytidine and the one patient treated with 2-chloro-2-deoxyadenosine. In 6 out of 7 patients, DNA methylation globally decreased by 8% after treatment. However, consistent microRNA upregulation was seen in mir-17-3p, mir-21, mir-29a, mir-29b, mir-29c, mir-30e, mir-104, mir-126, mir-128a, mir-130a, mir-141, mir-142-3p, mir-148a, mir-151, mir-199a, Mir-199a*, and mir-301 by real-time PCR using the Early Access Human Panel from Applied Biosystems. Using the University of California Santa Cruz genome database, mir-17, mir-126, mir-128a, mir-148a, mir-151, mir-199a, and mir-301 are sequence conserved in at least 5 mammalian species and are associated with a CpG island upstream from the predicted transcriptional start site. All but mir-148a are embedded within a known gene. No changes were seen in bcl-2, p53, mir-15, or mir-16. Qverexpression of mir-199-a and mir-199a* has been shown to induce cell cycle arrest. Mir-17-3p and mir-21 have anti-apoptotic properties. MiRNAs in patients with CLL are likely regulated by DNA promoter methylation.

1. A method for treating chronic lymphocytic leukemia (CLL) in a patient, comprising:
(a) determining a percentage of methylated cytosine relative to total cytosine in genomic DNA of leukemia cells of the patient;
(b) administering a treatment for CLL to the patient based on the determination.

2. The method of claim 1, further comprising determining a total white blood cell count for the patient.

3. The method of claim 1, further comprising determining whether the leukemia cells are positive for Zap-70.

4. The method of claim 1, further comprising determining whether the leukemia cells are positive for CD38.

5. The method of claim 1, further comprising determining whether the leukemia cells have a deletion in chromosome 13q.

6. The method of claim 1, further comprising determining whether the leukemia cells have a mutation in immunoglobulin heavy chain genes.

7. The method of claim 1, further comprising determining whether the leukemia cells comprise a higher proportion of methylated histone proteins relative to cells from a normal individual.

8. The method of claim 7, wherein the methylated histone proteins comprise methylated H3.

9. The method of claim 1, further comprising measuring expression of one or more micro RNAs (miRNAs) in the leukemia cells.

10. The method of claim 1, further comprising determining whether the leukemia cells comprise one or more methylated tumor suppressor genes.

11. The method of claim 1, further comprising determining whether the leukemia cells comprise a methylated gene selected from the group consisting of p15, p16, hMLH-1, MAGE-1, Twist2, Zap-70, CDH1, CDH13, DAPK, CRBP1, RARU, DLEU7, LEU1, LEU2, LEU5, KPNA3, CLLD6, CLLD7, and CLLD8.

12. The method of claim 10 or 11, comprising determining whether the tumor suppressor gene has a methylated promoter.

13. The method of claim 1, further comprising measuring beta-2-microglobulin in the leukemia cells.

14. The method of claim 1, wherein the treatment comprises administering an inhibitor of DNA methylation.

15. The method of claim 14, wherein the inhibitor is an inhibitor of Sadenosylhomocysteine hydrolase.

16. The method of claim 15, wherein the inhibitor comprises cladribine.

17. The method of claim 1, wherein the treatment comprises administering cladribine at a dose of about 0.05-0.1 mg/kg per day for no more than about 5 consecutive days.

18. The method of claim 14, wherein the inhibitor comprises and inhibitor of a DNA methylatransferase.

19. The method of claim 18, wherein the DNA methyltransferase is DNA methyltransferase I or DNA methyltransferase Mb.

20. The method of claim 14, wherein the inhibitor is selected from the group consisting of 2-chlorodeoxyadenosine, 5′-azacytidine, 5′-aza-2′-deoxycytidine, and mixtures thereof

21. The method of claim 1, wherein treatment is administered if the percentage is at least about 3.7%.

22. The method of claim 1, further comprising comparing the determined percentage to an expected percentage for cells obtained from one or more normal individuals having an age within five (5) years of the patient.

23. The method of claim 1, further comprising comparing the determined percentage to an expected percentage calculated using the formula E=4.00−0.0034×A, where A is the age of the patient and administering treatment if the determined percentage is greater than the expected percentage.

24. The method of claim 1, wherein treatment is administered if at least one of the following conditions is met:
(a) the patient has an age of 40-49 and the determined percentage is at least 3.91%;
(b) the patient has an age of 50-59 and the determined percentage is at least 3.82%;
(c) the patient has an age of 60-69 and the determined percentage is at least 3.82%
(d) the patient has an age of 70-79 and the determined percentage is at least 3.72%;
(e) the patient has an age of 80-89 and the determined percentage is at least 3.71%; and
(f) the patient has an age of 90-99 and the determined percentage is at least 3.63%.

25. The method of claim 1, wherein the method achieves at least about a 50% reduction in circulating leukemia cells.

26. A method for treating leukemia in a patient, comprising:
(a) determining a percentage of methylated cytosine relative to total cytosine in genomic DNA of leukemia cells of the patient;
(b) administering a treatment for leukemia to the patient based on the determination.

27. The method of claim 26, further comprising comparing the determined percentage to an expected percentage calculated using the formula E=4.00−0.0034×A, where A is the age of the patient and treatment is administered if the determined percentage is greater than the expected percentage.

28. The method of claim 26, wherein the leukemia is CLL.

29. The method of claim 26, wherein administering a treatment comprises administering a DNA methylation inhibitor.

30. The method of claim 26, wherein the treatment comprises administering cladribine.

31. A method for obtaining a prognosis for a patient having leukemia, the method comprising determining a percentage of methylated cytosine relative to total cytosine in genomic DNA of leukemia cells of the patient.

32. The method of claim 31, wherein the leukemia is CLL.

33. The method of claim 31, wherein the prognosis is an aggressive form of CLL.

34. Use of an inhibitor of DNA methylation for treating leukemia in a patient, wherein leukemia cells of the patient have an elevated percentage of methylated cytosine relative to total cytosine in genomic DNA when compared to an expected percentage.

35. Use of an inhibitor of DNA methylation for treating leukemia in a patient according to claim 34, wherein the expected percentage is calculated using the formula E=4.00−0.0034×A, wherein A is the age of the patient.

36. Use of an inhibitor of DNA methylation for treating leukemia in a patient according to claim 34, wherein the leukemia is CLL.

37. Use of an inhibitor of DNA methylation for treating leukemia in a patient according to claim 34, wherein the inhibitor comprises an inhibitor of S-adenosylhomocysteine hydrolase.

38. Use of an inhibitor of DNA methylation for treating leukemia in a patient according to claim 37, wherein the inhibitor comprises cladribine.


SEQUENCE LISTING (26 sequences)

SEQ ID NO: 1 - DNA, 22 bp, Artificial Sequence
  Description of Artificial Sequence Synthetic primer
ggaggaggttaaaggttttagt

SEQ ID NO: 2 - DNA, 21 bp, Artificial Sequence
  Description of Artificial Sequence Synthetic primer
aatccaaaaaaataaactccc

SEQ ID NO: 3 - DNA, 27 bp, Artificial Sequence
  Description of Artificial Sequence Synthetic primer
aggataatggaaggaaattaattagaa

SEQ ID NO: 4 - DNA, 26 bp, Artificial Sequence
  Description of Artificial Sequence Synthetic primer
aaccaacctttactacaaaacaaaat

SEQ ID NO: 5 - DNA, 25 bp, Artificial Sequence
  Description of Artificial Sequence Synthetic primer
tttgttttgtagtaaaggttggttt

SEQ ID NO: 6 - DNA, 26 bp, Artificial Sequence
  Description of Artificial Sequence Synthetic primer
tctaactccctcaatctcttattctt

SEQ ID NO: 7 - DNA, 25 bp, Artificial Sequence
  Description of Artificial Sequence Synthetic primer
ggaggaaggaaggatatagatttag

SEQ ID NO: 8 - DNA, 25 bp, Artificial Sequence
  Description of Artificial Sequence Synthetic primer
aatccctaatttaacccaatttcta

SEQ ID NO: 9 - DNA, 22 bp, Artificial Sequence
  Description of Artificial Sequence Synthetic primer
taggagtttgtgtggagtttgt

SEQ ID NO: 10 - DNA, 22 bp, Artificial Sequence
  Description of Artificial Sequence Synthetic primer
aaccaattaaaaattccacacc

SEQ ID NO: 11 - DNA, 22 bp, Artificial Sequence
  Description of Artificial Sequence Synthetic primer
gaggtagggttggatatttgag

SEQ ID NO: 12 - DNA, 23 bp, Artificial Sequence
  Description of Artificial Sequence Synthetic primer
caactaaaaccaaaacttccaac

SEQ ID NO: 13 - DNA, 19 bp, Artificial Sequence
  Description of Artificial Sequence Synthetic primer
ggaaaggggtgggattata

SEQ ID NO: 14 - DNA, 22 bp, Artificial Sequence
  Description of Artificial Sequence Synthetic primer
aaccacatactccatctacaaa

SEQ ID NO: 15 - DNA, 26 bp, Artificial Sequence
  Description of Artificial Sequence Synthetic primer
ggttggaagttttggttttagttgta

SEQ ID NO: 16 - DNA, 21 bp, Artificial Sequence
  Description of Artificial Sequence Synthetic primer
accctataatcccaccccttt

SEQ ID NO: 17 - DNA, 25 bp, Artificial Sequence
  Description of Artificial Sequence Synthetic primer
gagtaggtagtgtaggtttttaggt

SEQ ID NO: 18 - DNA, 22 bp, Artificial Sequence
  Description of Artificial Sequence Synthetic primer
taatcctctttccttttcaaat

SEQ ID NO: 19 - DNA, 20 bp, Artificial Sequence
  Description of Artificial Sequence Synthetic primer
gattaggtgggggttaggta

SEQ ID NO: 20 - DNA, 24 bp, Artificial Sequence
  Description of Artificial Sequence Synthetic primer
ctatttacttttatacaatccccc

SEQ ID NO: 21 - DNA, 22 bp, Artificial Sequence
  Description of Artificial Sequence Synthetic primer
gggttaggatttgttgtttttg

SEQ ID NO: 22 - DNA, 21 bp, Artificial Sequence
  Description of Artificial Sequence Synthetic primer
ccatactcccccctataactc

SEQ ID NO: 23 - DNA, 21 bp, Artificial Sequence
  Description of Artificial Sequence Synthetic primer
agttataggggggagtatggg

SEQ ID NO: 24 - DNA, 24 bp, Artificial Sequence
  Description of Artificial Sequence Synthetic primer
aaaaacccttacaaaaaaaaccac

SEQ ID NO: 25 - DNA, 24 bp, Artificial Sequence
  Description of Artificial Sequence Synthetic primer
gtggttttttttgtaagggttttt

SEQ ID NO: 26 - DNA, 20 bp, Artificial Sequence
  Description of Artificial Sequence Synthetic primer
acccctaacctcctctcaaa